(19) United States
(12) Patent Application Publication
(10) Pub. No.: US 2020/0377918 A1
(43) Pub. Date: Dec. 3, 2020

(54) PERFUSION MEDIUM

(71) Applicant: Boehringer Ingelheim International GmbH, Ingelheim am Rhein (DE)

(72) Inventors: Daisie OGAWA, Oakland, CA (US); Henry LIN, Westfield, NJ (US)

(21) Appl. No.: 16/496,523

(22) PCT Filed: Mar. 27, 2018
(86) PCT No.: PCT/EP2018/057749

§ 371 Date: Sep. 23, 2019

Related U.S. Application Data
(60) Provisional application No. 62/479,414, filed on Mar. 31, 2017.

(51) Int. Cl. C12P 21/00; C12N 5/07

(52) U.S. Cl. CPC C12P 21/00; C12N 5/06; C12N 2511/00; C12N 2500/24; C12N 2500/38; C12N 2500/90

(57) ABSTRACT

The invention relates to a method of culturing mammalian cells expressing a heterologous protein in a perfusion cell culture comprising adding iron and a retinoid to reduce wasteful cell bleed during production phase. The invention further relates to a serum-free perfusion medium comprising iron and a retinoid and its use for culturing cells in a perfusion culture during production phase or for reducing the cell bleed volume during production phase.

### TECHNICAL FIELD

The invention relates to a method of culturing mammalian cells expressing a heterologous protein in a perfusion cell culture comprising adding iron and a retinoid to reduce wasteful cell bleed during production phase. The invention further relates to a serum-free perfusion medium comprising iron and a retinoid and its use for culturing cells in a perfusion culture during production phase or for reducing the cell bleed volume during production phase.

### BACKGROUND

Three methods are typically used in commercial processes for the production of recombinant proteins by mammalian cell culture: batch culture, fed-batch culture, and perfusion culture.

Perfusion based methods offer potential improvement over the batch and fed-batch methods by adding fresh media and simultaneously removing spent media. Large scale commercial cell culture strategies may reach high cell densities of 60-90×10⁶ cells/mL, at which point about a third to over half of the reactor volume may be biomass. With perfusion based culture, extreme cell densities of >1×10⁸ cells/mL have been achieved. Typical perfusion cultures begin with a batch culture start-up lasting for a day or more to enable rapid initial cell growth and biomass accumulation, followed by continuous, step-wise and/or intermittent addition of fresh perfusion media to the culture and simultaneous removal of spent media with retention of cells throughout the growth and production phases of the culture. Various methods, such as sedimentation, centrifugation, or filtration, can be used to remove spent media, while maintaining the cells. Perfusion flow rates of a fraction of a working volume per day up to many multiple working volumes per day have been utilized.

Continuous processing for biologics manufacturing has numerous advantages over traditional fed batch, but many challenges still remain. Improvements in media have achieved higher viable cell densities (VCD) and, in turn, higher titers in perfusion processes. However, these elevated cell densities can become unsustainable resulting in viability loss and shortening of the perfusion run.

In the late 1970s and early 1980s modulation of cell growth by retinoids and their possible mechanism of action has been studied, because in some instances retinoids appeared to prevent or retard tumor growth. However, retinoids have not been studied as a supplement in mammalian cell culture for high performance protein production.

Haddox et al. (Cancer Research (1979) 38: 4930-4938) reports that vitamin A has prophylactic and therapeutic effects on growth of malignant and benign tumors. It discloses vitamin A as omithine decarboxylase induction inhibitor (ODC) in G1 phase in cell cycle synchronous CHO cultures in serum-containing medium. Further, low concentrations of vitamin A were disclosed to be growth stimulating. However, iron concentrations are not discussed and no advantageous effect of vitamin A in cell culture is implied.

Fischer et al. (The Journal of Cell biology (1981) 91: 373) report that retinol at concentrations of 10 to 50 μM had an inhibitory effect on growth and protein synthesis in CHO cultures and further that higher retinol concentrations were cytotoxic.

Jetten A M, (Federation Proceedings (1984) 43(1):134-139) reports that retinoids can enhance or inhibit cell growth in vitro. Due to its growth promoting activity it is suggested to be included in tests to optimize cell culture conditions in serum-free medium. Inhibition of growth by retinoids was observed in the presence of serum, but retinoids in serum-fee medium containing transferrin, insulin and EFG enhanced proliferation.

WO 2014/109858 A discloses the use of CDK4 inhibitor in cell culture such as batch, fed-batch and perfusion culture. A similar disclosure is found in Du et al. (Biotechnology and Bioengineering (2015) 112(1): 141-155). Du et al., further teaches that CDK4/6 inhibitors specifically inhibit the cell cycle without affecting other cellular targets. The application does not disclose vitamin A to improve cell culture performance.

WO 2016/006479 A discloses that a low L-asparagine concentration of 5 mM or less arrests cell growth in mammalian cell cultures, particularly in perfusion cultures and results in increased protein production. However, vitamin A is not mentioned in the application.

Vitamin A is a class of compounds comprising carotenoids and compounds having the biological activity of retinol (vitamin A alcohol). Of biological relevance are retinol and retinyl esters such as retinyl acetate and retinyl palmitate, which can be converted to the active metabolites retinal and retinoic acid in vivo. Vitamin A is classified as “generally regarded as safe” (GRAS) by the FDA and is routinely used in many chemically defined media. As such it has already been tested and hence no data regarding safety and clearance are required. While culture media are constantly altered and improved, there is still a need to improve perfusion culture by reducing waste and increasing productivity.

### SUMMARY OF THE INVENTION

A wasteful cell bleed can be utilized to maintain a sustainable VCD and preserve viability. In continuous processes a large proportion of the culture medium and hence product is lost due to the cell “bleed” which siphons off proliferating cells and medium in order to maintain a constant, sustainable viable cell density within the bioreactor. Up to one-third of harvestable material can be lost due to cell bleed. Using a cell bleed therefore decreases the product yield per run as the product within the cell bleed is not harvested. In order to decrease the cell bleed volume and thus retain more supernatant for harvest it is therefore advantageous to inhibit cell proliferation once the desired viable cell density is reached in the production phase. Thus, there is a need for controlling cell growth once an optimal VCD has been obtained and thus minimizing cell bleed to increase the product recovered per perfusion run and generating a more efficient method for operating perfusion processes.

In the present invention a perfusion culture medium comprising a retinoid and Fe ions and methods of culturing mammalian cells using said medium are provided. It is shown that vitamin A induced cell growth arrest in the presence of Fe ions of perfusion Chinese hamster ovary (CHO) cell cultures. This medium can inhibit proliferation without negatively affecting viability and/or cell specific productivity (qP). By controlling cell growth and thus minimizing cell bleed, the product recovered per perfusion run is greatly increased, generating a more efficient method for operating perfusion processes.

In one aspect a method of culturing mammalian cells expressing a heterologous protein in a perfusion cell culture is provided comprising: (a) culturing mammalian cells expressing a heterologous protein in a serum-free culture medium; (b) culturing the mammalian cells during growth phase by perfusion with a serum-free perfusion medium; and (c) maintaining the mammalian cells during production phase by perfusion with a serum-free perfusion medium comprising Fe ions at a concentration of 230 μM to 3.5 mM and a retinoid at a concentration of 10 μM to 400 μM, wherein step (b) is optional. In one embodiment of the method prior to step (c) the retinoid concentration in the mammalian cell culture is adjusted to the retinoid concentration in the serum-free perfusion medium of step (c), preferably by bolus addition.

In a related aspect a method of reducing cell bleeding in a perfusion cell culture expressing a heterologous protein is provided comprising: (a) culturing mammalian cells expressing a heterologous protein in a serum-free culture medium; (b) culturing the mammalian cells during growth phase by perfusion with a serum-free perfusion medium; and (c) maintaining the mammalian cells during production phase by perfusion with a serum-free perfusion medium comprising Fe ions at a concentration of 230 μM to 3.5 mM and a retinoid at a concentration of 10 μM to 400 μM, wherein step (b) is optional. In one embodiment of the method prior to step (c) the retinoid concentration in the mammalian cell culture is adjusted to the retinoid concentration in the serum-free perfusion medium of step (c), preferably by bolus addition.

In another related aspect a method of increasing protein production in a perfusion cell culture expressing a heterologous protein is provided comprising: (a) culturing mammalian cells expressing a heterologous protein in a serum-free culture medium; (b) culturing the mammalian cells during growth phase by perfusion with a serum-free perfusion medium; and (c) maintaining the mammalian cells during production phase by perfusion with a serum-free perfusion medium comprising Fe ions at a concentration of 230 μM to 3.5 mM and a retinoid ata concentration of 10 μM to 400 μM, wherein step (b) is optional. In one embodiment of the method prior to step (c) the retinoid concentration in the mammalian cell culture is adjusted to the retinoid concentration in the serum-free perfusion medium of step (c), preferably by bolus addition.

Step (c) is started once the target cell density is reached. Thus, it may be started at a cell density of 10×10⁶ cells/ml to about 120×10⁶ cells/ml or even higher. Preferably step (c) is initiated at a cell density of at least 10×10⁶ cells/ml, at least 20×10⁶ cells/ml, at least 30×10⁶ cells/ml, at least 40×10⁶ cells/ml or at least 50×10⁶ cells/. Most preferably step (c) is initiated at a cell density of about 30 to about 50×10⁶ cells/ml.

Step (c) of the methods of the invention may further comprise maintaining a cell density by cell bleeding. Using the methods of the present invention the cell bleeding is reduced compared to a perfusion cell culture using the same serum-free perfusion medium with Fe ions at a concentration of less than 230 μM and a retinoid at a concentration of less than 10 μM and cultured under the same conditions. In addition to the serum-free perfusion medium of step (c) also the serum-free perfusion medium in step (b), and optionally also the serum-free culture medium of step (a) may comprise Fe ions at a concentration of 230 μM to 3.5 mM.

In some embodiments of the methods of the invention the retinoid concentration is about 60 μM to 400 μM, preferably about 100 μM to about 200 μM and more preferably about 150 μM to about 200 μM. In some embodiments the Fe ion concentration is about 350 μM to 3.5 mM, preferably about 1 mM to 3.5 mM, more preferably about 1.4 mM to 3.5 mM and even more preferably about 1.6 mM to 3.5 mM or the Fe ion concentration is about 350 μM to about 3.1 mM, preferably about 1 mM to about 3.1 mM, more preferably about 1.4 mM to about 3.1 mM and even more preferably about 1.6 mM to about 3.1 mM.

The retinoid may be retinol, retinal, retinoic acid or a retinyl ester and is preferably retinol or a retinyl ester. The retinyl ester may be retinyl acetate or retinyl palmitate, preferably retinyl acetate. The Fe ion may be provided as FeSO₄, Fe-citrate, Fe-transferrin, iron choline citrate, Fe-EDTA, Fe(NO₃)₃, FeCl₂, FeCl₃, or combinations thereof, preferably as FeSO₄, Fe-citrate, Fe(NO₃)₃, Fe-transferrin, or combinations thereof, more preferably as Fe-citrate.

The osmolarity of the serum-free perfusion medium should be in the range of between 300 and 1400 mOsmol/kg, preferably between 300 and 500 mOsmol/kg, more preferably between 330 and 450 mOsmol/kg and even more preferably between 360 and 390 mOsmol/kg.

The mammalian cell may be a Chinese hamster ovary (CHO) cell, preferably a CHO-DG44 cell, a CHO-K1 cell, a CHO DXB11 cell, a CHO-S cell, a CHO GS deficient cell or a derivative of any of these cells.

The serum-free perfusion medium may be chemically defined and/or hydrolysate free. Preferably the serum-free perfusion medium is protein-free or protein-free except for recombinant insulin and/or insulin-like growth factor, more preferably the serum-free perfusion medium is chemically defined and protein-free or protein-free except for recombinant insulin and/or insulin-like growth factor.

In a further aspect a method of producing a therapeutic protein using the methods of the invention is provided optionally comprising a further step of purifying and formulating the therapeutic protein into a pharmaceutically acceptable formulation.

In another aspect a serum-free perfusion medium is provided comprising Fe ions at a concentration ranging from 230 μM to 3.5 mM and a retinoid in a concentration ranging from 10 μM to 400 μM. In some embodiments the retinoid concentration is about 60 μM to 400 μM, preferably about 100 μM to about 200 μM and more preferably about 150 μM to about 200 μM. In some embodiments the Fe ion concentration is about 350 μM to 3.5 mM, preferably about 1 mM to 3.5 mM, more preferably about 1.4 mM to 3.5 mM and even more preferably about 1.6 mM to 3.5 mM or the Fe ion concentration is about 350 μM to about 3.1 mM, preferably about 1 mM to about 3.1 mM, more preferably about 1.4 mM to about 3.1 mM and even more preferably about 1.6 mM to about 3.1 mM.

The retinoid may be retinol, retinal, retinoic acid or a retinyl ester and is preferably retinol or a retinyl ester. The retinyl ester may be retinyl acetate or retinyl palmitate, preferably retinyl acetate. The Fe ion may be provided as FeSO₄, Fe-citrate, Fe-transferrin, iron choline citrate, Fe-EDTA, Fe(NO₃)₃, FeCl₂, FeCl₃, or combinations thereof, preferably as FeSO₄, Fe-citrate, Fe(NO₃)₃, Fe-transferrin, or combinations thereof, more preferably as Fe-citrate.

The osmolarity of the serum-free perfusion medium should be in the range of between 300 and 1400 mOsmol/kg, preferably between 300 and 500 mOsmol/kg, more preferably between 330 and 450 mOsmol/kg and even more preferably between 360 and 390 mOsmol/kg.

The serum-free perfusion medium may be chemically defined and/or hydrolysate free. Preferably the serum-free perfusion medium is protein-free or protein-free except for recombinant insulin and/or insulin-like growth factor, more preferably the serum-free perfusion medium is chemically defined and protein-free or protein-free except for recombinant insulin and/or insulin-like growth factor.

In yet another aspect a use of the serum-free perfusion medium of the invention for culturing mammalian cells in a perfusion culture during production phase or for reducing the total cell bleed volume in a perfusion culture is provided. Alternatively a use of the serum-free perfusion medium of the invention for increasing protein production in a perfusion cell culture is provided.

### BRIEF DESCRIPTION OF THE DRAWINGS

FIG. 1: CHO cells in small scale culture were exposed to control (diamonds), ethanol control (squares), 5 μM retinyl acetate (triangles) and 20 μM retinyl acetate (X) using a basal medium containing 3.1 mM ferric citrate. Shown in panel (A) is the viable cell density (e5 cells/mL, which is the technical spelling for “10⁵ cells/mL”) and in panel (B) the viability of CHO cells in the culture. Data represent average of triplicates; error bars represent standard deviation.

FIG. 2: CHO cells in small scale culture were exposed to control (diamonds), ethanol control (squares), 40 μM retinyl acetate (triangles), 60 μM retinyl acetate (X), 80 μM retinyl acetate (open circles), and 100 μM retinyl acetate (closed circles) using a basal medium containing 3.1 mM ferric citrate. Shown in panel (A) is the viable cell density (e5 cells/mL) and in panel (B) the viability of CHO cells in the culture. Data represent average of triplicates; error bars represent standard deviation.

FIG. 3: CHO cells in small scale culture were exposed to control (diamonds), ethanol control (squares), 200 μM retinyl acetate (X), 400 μM retinyl acetate (open circles), and 600 μM retinyl acetate (closed circles) using a basal medium containing 3.1 mM ferric citrate. Shown in panel (A) is the viable cell density (VCD e5 cells/mL) and in panel (B) the viability of CHO cells in the culture. Data represent average of triplicates; error bars represent standard deviation.

FIG. 4: CHO cells cultured in a bioreactor using perfusion culture were cultured from days 15 to 20 in a medium containing 1.6 mM ferric citrate and a) control, no vitamin (squares), b) 50 μM retinyl acetate (circles), and c) 100 μM retinyl acetate (triangles), with a successive treatment from days 20-23 with a) control medium (squares), b) 150 μM retinyl acetate (circles), and c) 200 μM retinyl acetate (triangles). Shown in panel (A) is the viable cell density (VCD e5 cells/mL) and in panel (B) the viability of CHO cells in the culture.

FIG. 5. CHO cells cultured in a bioreactor using perfusion culture were cultured from days 15 to 20 in a medium containing 1.6 mM ferric citrate and a) control, no vitamin (squares), b) 50 μM retinyl acetate (circles), and c) 100 μM retinyl acetate (triangles), with a successive treatment from days 20-24 with a) control medium (squares), b) 150 μM retinyl acetate (circles), and c) 200 μM retinyl acetate (triangles, only until day 23). Shown in panel (A) is the bleed flow rates (g/min)), and in panel (B) a sector representing the normalized cell bleed rate on days 20 to 24 of CHO cell cultures.

FIG. 6: CHO cells cultured in a bioreactor using perfusion culture were cultured from days 25 to 29 in a medium containing 350 μM ferric citrate (triangle) or 350 μM ferric citrate and 100 μM retinyl acetate (squares). Shown in panel (A) is the viable cell density (VCD e5 cells/mL) and in panel (B) the viability of CHO cells in the perfusion culture determined by Trypan blue dye exclusion in the perfusion culture.

FIG. 7: CHO cells cultured in a bioreactor using perfusion culture were cultured from days 25 to 29 in a medium containing 350 μM ferric citrate (triangle) or 350 μM ferric citrate and 100 μM retinyl acetate (squares). Shown in (A) is the relative cell bleed rate of the bioreactor and in (B) the specific growth rate of CHO perfusion culture. Values are normalized to levels at the start of respective treatments on day 25.

FIG. 8: Viability comparison between low Fe-citrate (230 μM; open symbols) and high Fe-citrate concentrations (3.1 mM; closed symbols) for retinyl acetate concentrations of 200 μM (squares), 400 μM (circles), and 500 μM (triangles) in small scale culture.

### DETAILED DESCRIPTION

The general embodiments “comprising” or “comprised” encompass the more specific embodiment “consisting of”. Furthermore, singular and plural forms are not used in a limiting way. As used herein, the singular forms “a”, “an” and “the” designate both the singular and the plural, unless expressly stated to designate the singular only.

The term “perfusion” as used herein refers to maintaining a cell culture bioreactor in which equivalent volumes of media are simultaneously added and removed from the reactor while the cells are retained in the reactor. A perfusion culture may also be referred to as continuous culture. This provides a steady source of fresh nutrients and constant removal of cell waste products. Perfusion is commonly used to attain much higher cell density and thus a higher volumetric productivity than conventional bioreactor batch or fed batch conditions. Secreted protein products can be continuously harvested while retaining the cells in the reactor, e.g., by filtration, alternating tangential flow (ATF), cell sedimentation, ultrasonic separation, hydrocyclones, or any other method known to the person skilled in the art or as described Kompala and Ozturk (Cell Culture Technology for Pharmaceutical and Cell-Based Therapies, (2006), Taylor & Francis Group, LLC, pages 387-416). Mammalian cells may be grown in suspension cultures (homogeneous cultures) or attached to surfaces or entrapped in different devices (heterogeneous cultures). In order to keep the working volume in the bioreactor constant the harvest rate and cell bleed (fluid removal) should be equal to the predetermined perfusion rate. The culture is typically initiated by a batch culture and the perfusion is started on day 2-3 after inoculation when the cells are still in exponential growth phase and before nutrient limitation occurs.

Perfusion based methods offer potential improvement over the batch and fed-batch methods by adding fresh media and simultaneously removing spent media. Large scale commercial cell culture strategies may reach high cell densities of 60-90×10⁶ cells/mL, at which point about a third to over half of the reactor volume may be biomass. With perfusion based culture, extreme cell densities of >1×10⁸ cells/mL have been achieved. Typical perfusion cultures begin with a batch culture start-up lasting for a day or more to enable rapid initial cell growth and biomass accumulation, followed by continuous, step-wise and/or intermittent addition of fresh feed media to the culture and simultaneous removal of spent media with retention of cells throughout the growth and production phases of the culture. Various methods, such as sedimentation, centrifugation, or filtration, can be used to remove spent media, while maintaining the cells. Perfusion flow rates of a fraction of a working volume per day up to many multiple working volumes per day have been utilized.

The term “perfusion rate” as used herein is the volume added and removed and is typically measured per day. It depends on the cell density and the medium. It should be minimized to reduce the dilution of the product of interest, i.e., harvest titer, while ensuring adequate rates of nutrient addition and by-product removal. Perfusion is typically started on day 2-3 after inoculation when the cells are still in the exponential growth phase and hence perfusion rate may be increased over the culture. Increase in perfusion rate may be incremental or continuously, i.e., based on cell density or nutrient consumption. It typically starts with 0.5 or 1 vessel volume per day (VVD) and may go up to about 5 VVD. Preferably, the perfusion rate is between 0.5 to 2 VVD. The increase may be by 0.5 to 1 VVD. For continuous increase in perfusion, a biomass probe may be interfaced with the harvest pump, such that the perfusion rate is increased as a linear function of the cell density determined by the biomass probe, based on a desired cell specific perfusion rate (CSPR). The CSPR equals the perfusion rate per cell density and an ideal CSPR depend on the cell line and the cell medium. The ideal CSPR should result in optimal growth rate and productivity. A CSPR of 50 to 100 pL/cell per day may be a reasonable starting range, which can be adjusted to find the optimal rate for a specific cell line.

The term “steady state” as used herein refers to the condition where cell density and bioreactor environment remain relatively constant. This can be achieved by cell bleeding, nutrient limitation and/or temperature reduction. In most perfusion cultures nutrient supply and waste removal will allow for constant cell growth and productivity and cell bleeding is required to maintain a constant viable cell density or to maintain the cells in steady state. A typical viable cell density at steady state is 10 to 50e6 cells/ml. The viable cell density may vary depending on the perfusion rate. A higher cell density can be reached by increasing the perfusion rate or by optimizing the medium for use with perfusion. At a very high viable cell density perfusion cultures become difficult to control within a bioreactor.

The terms “cell bleed” and “cell bleeding” are used interchangeably herein and refer to the removal of cells and medium from the bioreactor in order to maintain a constant, sustainable viable cell density within the bioreactor. The constant, sustainable viable cell density may also be referred to as target cell density. This cell bleed may be done using a dip tube and a peristaltic pump at a defined flow rate. The tubing should have the right size with a too narrow tube being prone to cell aggregation and clogging while if too large the cells may settle. The cell bleed can be determined based on growth rate, thus viable cell density can be limited to a desired volume in a continuous manner. Alternatively, cells may be removed at a certain frequency, e.g., once a day, and replaced by media to maintain cell density within a predictable range. Ideally the cell bleed rate is equal to the growth rate to maintain a steady cell density.

Typically the product of interest removed with the cell bleed is discarded and therefore lost for the harvest. Opposite to a permeate, the cell bleed contains cells, which makes storage of the product prior to purification more difficult and can have detrimental effects on product quality. Thus, the cells would have to be removed continuously prior to storage and product purification, which would be laborious and cost inefficient. For slow growing cells the cell bleed may be about 10% of the removed fluid and for fast growing cells the cell bleed may be about 30% of the removed fluid. Thus, the product loss through the cell bleed may be about 30% of the product produced in total. The “permeate” as used herein refers to the harvest from which the cells have been separated to be retained in the culture vessel.

The term “culture” or “cell culture” is used interchangeably and refer to a cell population that is maintained in a medium under conditions suitable to allow survival and/or growth of the cell population. The present invention only relates to mammalian cell cultures. Mammalian cells may be cultured in suspension or while attached to a solid support. As will be clear to the person skilled a cell culture refers to a combination comprising the cell population and the medium in which the population is suspended.

The term “culturing” as used herein refers to a process by which mammalian cells are grown or maintained under controlled conditions and under conditions that supports growth and/or survival of the cells. The term “maintaining cells” as used herein is used interchangeably with “culturing cells”. Culturing may also refer to a step of inoculating cells in a culture medium.

As used herein, the term “batch culture” is a discontinuous method where cells are grown in a fixed volume of culture media for a short period of time followed by a full harvest. Cultures grown using the batch method experience an increase in cell density until a maximum cell density is reached, followed by a decline in viable cell density as the media components are consumed and levels of metabolic by-products (such as lactate and ammonia) accumulate. Harvest typically occurs at or soon after the point when the maximum cell density is achieved (typically 5-10×10⁶ cells/mL, depending on media formulation, cell line, etc.) typically around 3 to 7 days.

As used herein, the term “fed-batch culture” improves on the batch process by providing bolus or continuous media feeds to replenish those media components that have been consumed. Since fed-batch cultures receive additional nutrients throughout the culture process, they have the potential to achieve higher cell densities (>10 to 30×10⁶ cells/ml, depending on media formulation, cell line, etc.) and increased product titers, when compared to the batch method. Unlike the batch process, a biphasic culture can be created and sustained by manipulating feeding strategies and media formulations to distinguish the period of cell proliferation to achieve a desired cell density (the growth phase) from the period of suspended or slow cell growth (the production phase). As such, fed batch cultures have the potential to achieve higher product titers compared to batch cultures. As with the batch method, metabolic by-product accumulation will lead to declining cell viability over time as these progressively accumulate within the cell culture media, which limits the duration of the production phase to about 1.5 to 3 weeks. Fed-batch cultures are discontinuous and harvest typically occurs when metabolic by-product levels or the culture viability reach predetermined levels.

The term “polypeptide” or “protein” is used interchangeably herein with “amino acid residue sequences” and refers to a polymer of amino acids. These terms also include proteins that are post-translationally modified through reactions that include, but are not limited to, glycosylation, acetylation, phosphorylation or protein processing. Modifications and changes, for example fusions to other proteins, amino acid sequence substitutions, deletions or insertions, can be made in the structure of a polypeptide while the molecule maintains its biological functional activity. For example certain amino acid sequence substitutions can be made in a polypeptide or its underlying nucleic acid coding sequence and a protein can be obtained with the same properties. The terms also apply to amino acid polymers in which one or more amino acid residue is an analog or mimetic of a corresponding naturally occurring amino acid. The term “polypeptide” typically refers to a sequence with more than 10 amino acids and the term “peptide” to sequences with up to 10 amino acids in length.

The term “heterologous protein” as used herein refers to a polypeptide derived from a different organism or a different species from the host cell. The heterologous protein is coded for by a heterologous polynucleotide that is experimentally put into the host cell that does not naturally express that protein. A heterologous polynucleotide may also be referred to as transgene. Thus, it may be a gene or open reading frame (ORF) coding for a heterologous protein. The term “heterologous” when used with reference to a protein may also indicate that the protein comprises amino acid sequences that are not found in the same relationship to each other or the same length in nature. Thus, it also encompasses recombinant proteins. Heterologous may also refer to a polynucleotide sequence, such as a gene or transgene, or a portion thereof, being inserted into the mammalian cell's genome in a location in which it is not typically found. In the present invention the heterologous protein is preferably a therapeutic protein.

The term “medium”, “cell culture medium” and “culture medium” are used interchangeably herein and refer to a solution of nutrients that nourish cells, particularly mammalian cells. Cell culture media formulations are well known in the art. Typically a cell culture medium provides essential and non-essential amino acids, vitamins, energy sources, lipids and trace elements required by the cell for minimal growth and/or survival, as well as buffers, and salts. A culture medium may also contain supplementary components that enhance growth and/or survival above the minimal rate, including, but not limited to, hormones and/or other growth factors, particular ions (such as sodium, chloride, calcium, magnesium, and phosphate), buffers, vitamins, nucleosides or nucleotides, trace elements (inorganic compounds usually present at very low final concentrations), amino acids, lipids, and/or glucose or other energy source; as described herein. In certain embodiments, a medium is advantageously formulated to a pH and salt concentration optimal for cell survival and proliferation. The medium according to the invention is a perfusion culture medium that is added after the beginning of the cell culture. In certain embodiments, the cell culture medium is a mixture of a starting nutrient solution (basal medium or inoculation medium) and any culture medium that is added after the beginning of the cell culture.

The term “serum-free” as used herein refers to a cell culture medium that does not contain animal or human serum, such as fetal bovine serum. Preferably serum-free medium is free of proteins isolated from any animal or human derived serum. Various tissue culture media, including defined culture media, are commercially available, for example, any one or a combination of the following cell culture media can be used: RPMI-1640 Medium, RPMI-1641 Medium, Dulbecco's Modified Eagle's Medium (DMEM), Minimum Essential Medium Eagle, F-12K Medium, Ham's F12 Medium, Iscove's Modified Dulbecco's Medium, McCoy's 5A Medium, Leibovitz's L-15 Medium, and serum-free media such as EX-CELL™ 300 Series (JRH Biosciences, Lenexa, Kansas), among others. Serum-free versions of such culture media are also available. Cell culture media may be supplemented with additional or increased concentrations of components such as amino acids, salts, sugars, vitamins, hormones, growth factors, buffers, antibiotics, lipids, trace elements and the like, depending on the requirements of the cells to be cultured and/or the desired cell culture parameters.

The term “protein-free” as used herein refers to a cell culture medium that does not contain any protein. Thus, it is devoid of proteins isolated from an animal or human, derived from serum or recombinantly produced proteins, such as recombinant proteins produced in mammalian, bacterial, insect or yeast cells. A protein-free medium may contain single recombinant proteins, such as insulin or insulin-like growth factor, but only if this addition is explicitly stated.

As used herein the term “chemically defined” refers to a culture medium, which is serum-free and which does not contain any hydrolysates, such as protein hydrolysates derived from yeast, plants or animals. Preferably a chemically defined medium is also protein-free or contains only selected recombinantly produced (not animal derived) proteins, such as insulin or insulin-like growth factor. Chemically defined medium consist of a mixture of characterized and purified substances. An example of a chemically defined medium is for example CD-CHO medium from Invitrogen (Carlsbad, Calif., US).

The term “suspension cells” or “non-adherent cells” as used herein relates to cells that are cultured in suspension in liquid medium. Adhesive cells such as CHO cells may be adapted to be grown in suspension and thereby lose their ability to attach to the surface of the vessel or tissue culture dish.

As used herein, the term “bioreactor” means any vessel useful for the growth of a cell culture. A bioreactor can be of any size as long as it is useful for the culturing of cells; typically, a bioreactor is sized appropriate to the volume of cell culture being grown inside of it. Typically, a bioreactor will be at least 1 liter and may be 2, 5, 10, 50, 100, 200, 250, 500, 1,000, 1,500, 2,000, 2,500, 5,000, 8,000, 10,000, 12,000 liters or more, or any volume in between. The internal conditions of the bioreactor, including, but not limited to pH and temperature, can be controlled during the culturing period. Those of ordinary skill in the art will be aware of, and will be able to select, suitable bioreactors for use in practicing the present invention based on the relevant considerations. The cell cultures used in the methods of the present invention can be grown in any bioreactor suitable for perfusion culture.

As used herein, “cell density” refers to the number of cells in a given volume of culture medium. “Viable cell density” refers to the number of live cells in a given volume of culture medium, as determined by standard viability assays (such as trypan blue dye exclusion method).

As used herein, the term “cell viability” means the ability of cells in culture to survive under a given set of culture conditions or experimental variations. The term as used herein also refers to that portion of cells which are alive at a particular time in relation to the total number of cells, living and dead, in the culture at that time.

As used herein, the term “titer” means the total amount of a polypeptide or protein of interest (which may be a naturally occurring or recombinant protein of interest) produced by a cell culture in a given amount of medium volume. Titer can be expressed in units of milligrams or micrograms of polypeptide or protein per milliliter (or other measure of volume) of medium.

As used herein, the term “yield” refers to the amount of heterologous protein produced in perfusion culture over a certain period of time. The “total yield” refers to the amount of heterologous protein produced in perfusion culture over the entire run.

The term “reduction”, “reduced” or “reduce”, as used herein, generally means a decrease by at least 10% as compared to a reference level, for example a decrease by at least about 20%, or at least about 30%, or at least about 40%, or at least about 50%, or at least about 60%, or at least about 70%, or at least about 75%, or at least about 80%, or at least about 90% or up to and including a 100% decrease, or any integer decrease between 10-100% as compared to a control mammalian cell culture, which is cultured under the same conditions using the same serum-free perfusion medium without Fe ions and a retinoid at the concentrations used in the perfusion medium of the invention.

The term “enhancement”, “enhanced”, “enhanced”, “increase” or “increased”, as used herein, generally means an increase by at least 10% as compared to a control cell, for example an increase by at least about 20%, or at least about 30%, or at least about 40%, or at least about 50%, or at least about 75%, or at least about 80%, or at least about 90%, or at least about 100%, or at least about 200%, or at least about 300%, or any integer decrease between 10-300% as compared to a mammalian cell culture, which is cultured under the same conditions using the same serum-free perfusion medium without Fe ions and a retinoid at the concentrations used in the perfusion medium of the invention.

As used herein, a “control cell culture” or “control mammalian cell culture” is a cell which is the same as the cell culture to which it is compared to, except that the perfusion medium does not have the Fe ion and retinoid concentrations of the perfusion medium of the invention.

The term “mammalian cells” as used herein are cells lines suitable for the production of a heterologous protein, preferably a therapeutic protein, more preferably a secreted recombinant therapeutic protein. Preferred mammalian cells according to the invention are rodent cells such as hamster cells. The mammalian cells are isolated cells or cell lines. The mammalian cells are preferably transformed and/or immortalized cell lines. They are adapted to serial passages in cell culture and do not include primary non-transformed cells or cells that are part of an organ structure. Preferred mammalian cells are BHK21, BHK TK⁻, CHO, CHO-K1, CHO-S cells, CHO-DXB11 (also referred to as CHO-DUKX or DuxB11), and CHO-DG44 cells or the derivatives/progenies of any of such cell line. Particularly preferred are CHO-DG44, CHO-K1 and BHK21, and even more preferred are CHO-DG44 and CHO-K1 cells. Most preferred are CHO-DG44 cells. Glutamine synthetase (GS)-deficient derivatives of the mammalian cell, particularly of the CHO-DG44 and CHO-K1 cell are also encompassed. The mammalian cell may further comprise one or more expression cassette(s) encoding a heterologous protein, preferably a recombinant secreted therapeutic protein. The mammalian cells may also be murine cells such as murine myeloma cells, such as NSO and Sp2/0 cells or the derivatives/progenies of any of such cell line. However, derivatives/progenies of those cells, other mammalian cells, including but not limited to human, mice, rat, monkey, and rodent cell lines, can also be used in the present invention, particularly for the production of biopharmaceutical proteins.

The term “retinoid” as used herein refers to a class of chemical compounds that are related in structure and biological function to retinol (vitamin A), more specifically to the group of compounds consisting of retinol, retinal, retinoic acid and a retinyl ester such as retinyl acetate or retinyl palmitate. Particularly preferred for use in the present invention are retinol and retinyl esters, even more preferred are retinyl esters such as retinyl acetate or retinyl palm itate, wherein retinyl acetate is preferred.

The term “growth phase” as used herein refers to the phase of cell culture where the cells proliferate exponentially and viable cell density in the bioreactor is increasing. Cells in culture usually proliferate following a standard growth pattern. The first phase of growth after the culture is seeded is the lag phase, which is a period of slow growth when the cells are adapting to the culture environment and preparing for fast growth. The lag phase is followed by the growth phase (also referred to as log phase or logarithmic phase), a period where the cells proliferate exponentially and consume the nutrients of the growth medium. The “production phase” starts once the target cell density is reached and/or harvest is started. A typical target cell density is in the range of 10×10⁶ cells/ml to about 120×10⁶ cells/ml, but may be even higher. Thus, the target cell density according to the present invention is at least 10×10⁶ cells/ml, at least 20×10⁶ cells/ml, at least 30×10⁶ cells/ml, at least 40×10⁶ cells/ml or at least 50×10⁶ cells/ml. Most preferably the target cell density is about 30 to about 50×10⁶ cells/ml. Viable cell density is dependent on the perfusion rate and can be maintained at a constant level using regular or continuous cell bleeds.

The term “growth-arrest” as used herein refers to cells that are stopped from increasing in number, i.e., from cell division. The cell cycle comprises the interphase and the mitotic phase. The interphase consists of three phases: DNA replication is confined to S phase; G₁ is the gap between M phase and S phase, while G₂ is the gap between S phase and M phase. In M phase, the nucleus and then the cytoplasm divide. In the absence of a mitogenic signal to proliferate or in the presence of compounds that induce growth arrest the cell cycle arrests. The cells may partly disassemble their cell-cycle control system and exit from the cycle to a specialized, non-dividing state called G₀.

The term “bolus addition” as used herein refers to an addition that immediately adjusts the concentration in the cell culture to the desired concentration. According to the invention it means that the retinoid concentration in the cell culture is instantaneously adjusted to the retinoid concentration of the invention. This is to avoid a transitional phase wherein cells are cultured at a lower retinoid concentration that may result in unwanted proliferative activity.

The term “about” as used herein refers to a variation around the actual value provided and encompasses plus and minus 10% of the value.

Methods of Culturing Cells using Perfusion Culture

For the purposes of understanding, yet without limitation, it will be appreciated by the skilled practitioner that cell cultures and culturing runs for protein production can include three general types; namely, perfusion culture, batch culture and fed-batch culture. In a perfusion culture, for example, fresh culture medium supplement is provided to the cells during the culturing period, while old culture medium is removed daily and the product is harvested, for example, daily or continuously. In perfusion culture, perfusion medium can be added daily and can be added continuously, i.e., as a drip or infusion. For perfusion culturing, the cells can remain in culture as long as is desired, so long as the cells remain alive and the environmental and culturing conditions are maintained. Since the cells grow continuously, it is typically required to remove cells during the run in order to maintain a constant viable cell density, which is referred to as cell bleed. The cell bleed contains product in the culture medium removed with the cells, which is typically discarded and hence wasted. Thus, maintaining the viable cell density during production phase without or with only minimal cell bleeding is advantageous and increases the total yield per run.

In batch culture, cells are initially cultured in medium and this medium is not removed, replaced, or supplemented, i.e., the cells are not “fed” with new medium, during or before the end of the culturing run. The desired product is harvested at the end of the culturing run.

For fed-batch cultures, the culturing run time is increased by supplementing the culture medium one or more times daily (or continuously) with fresh medium during the run, i.e., the cells are “fed” with new medium (“feeding medium”) during the culturing period. Fed-batch cultures can include the various feeding regimens and times as described above, for example, daily, every other day, every two days, etc., more than once per day, or less than once per day, and so on. Further, fed-batch cultures can be fed continuously with feeding medium. The desired product is then harvested at the end of the culturing/production run.

According to the present invention mammalian cells are cultured in perfusion culture. During heterologous protein production it is desirable to have a controlled system where cells are grown to a desired viable cell density and then the cells are switched to a growth-arrested, high productivity state where the cells use energy and substrates to produce the heterologous protein of interest rather than cell growth and cell division. Methods for accomplishing this goal, such as temperature shifts and amino acid starvation, are not always successful and can have undesirable effects on product quality. As described herein viable cell density during production phase can be maintained at a desirable level by performing a regular cell bleed. However, this results in discarding heterologous protein of interest. Cell growth-arrest during production phase results in a reduced need for a cell bleed and may even maintain cells in a more productive state.

Provided herein is a method of culturing mammalian cells expressing a heterologous protein in a perfusion cell culture comprising: (a) culturing mammalian cells expressing a heterologous protein in a serum-free culture medium; (b) culturing the mammalian cells during growth phase by perfusion with a serum-free perfusion medium; and (c) maintaining the mammalian cells during production phase by perfusion with a serum-free perfusion medium comprising Fe ions at a concentration of 230 μM to 3.5 mM and a retinoid at a concentration of 10 μM to 400 μM, wherein step (b) is optional. In one embodiment of the method prior to step (c) the retinoid concentration in the mammalian cell culture is adjusted to the retinoid concentration in the serum-free perfusion medium of step (c), preferably by bolus addition.

Also provided herein is a method of reducing cell bleeding in a perfusion cell culture and/or increasing protein production in a perfusion cell culture expressing a heterologous protein comprising: (a) culturing mammalian cells expressing a heterologous protein in a serum-free culture medium; (b) culturing the mammalian cells during growth phase by perfusion with a serum-free perfusion medium; and (c) maintaining the mammalian cells during production phase by perfusion with a serum-free perfusion medium comprising Fe ions ata concentration of 230 μM to 3.5 mM and a retinoid ata concentration of 10 μM to 400 μM, wherein step (b) is optional. In one embodiment of the method prior to step (c) the retinoid concentration in the mammalian cell culture is adjusted to the retinoid concentration in the serum-free perfusion medium of step (c), preferably by bolus addition.

It is also encompassed by the invention that the perfusion culture is inoculated with a very high cell density and perfusion is started immediately or shortly after inoculation of mammalian cells expressing a heterologous protein in a serum-free culture medium. Further, step (b) culturing the mammalian cells during growth phase by perfusion with a serum-free perfusion medium may be optional so that the mammalian cells are immediately cultured according to step (c) during production phase by perfusion with a serum-free perfusion medium comprising Fe ions at a concentration of 230 μM to 3.5 mM and a retinoid at a concentration of 10 μM to 400 μM. Preferably, prior to step (c) the retinoid concentration in the mammalian cell culture is adjusted to the retinoid concentration in the serum-free perfusion medium of step (c), preferably by bolus addition.

Thus, also provided herein is a method of culturing mammalian cells expressing a heterologous protein and/or reducing cell bleeding in a perfusion cell culture and/or increasing protein production in a perfusion cell culture expressing a heterologous protein comprising: (a) inoculating mammalian cells expressing a heterologous protein in a serum-free culture medium; (b) optionally culturing the mammalian cells during growth phase by perfusion with a serum-free perfusion medium; and (c) maintaining the mammalian cells during production phase by perfusion with a serum-free perfusion medium comprising Fe ions at a concentration of 230 μM to 3.5 mM and a retinoid at a concentration of 10 μM to 400 μM. In a preferred embodiment of the methods of the invention prior to step (c) the retinoid concentration in the mammalian cell culture is adjusted to the retinoid concentration in the serum-free perfusion medium of step (c) by bolus addition. Increasing the protein production encompasses an increased protein product yield over the perfusion run or over a certain period of time. It also encompasses an increased specific protein production per cell.

According to the methods of the invention, culturing the mammalian cells in step (a) may be limited to inoculating mammalian cells expressing a heterologous protein in a serum-free medium and hence does not need to include an actual culturing step prior to the start of perfusion. Further according to the methods of the invention, maintaining the mammalian cells during production phase by perfusion includes culturing the mammalian cells during production phase by perfusion at a constant viable cell density.

The production phase starts once the target cell density is reached. Preferably step (c) is started once the target cell density is reached. It may be started ata cell density of 10×10⁶ cells/ml to about 120×10⁶ cells/ml or even higher. Preferably step (c) is initiated at a cell density of at least 10×10⁶ cells/ml, at least 20×10⁶ cells/ml, at least 30×10⁶ cells/ml, at least 40×10⁶ cells/ml or at least 50×10⁶ cells/ml. Most preferably step (c) is initiated at a cell density of about 30 to about 50×10⁶ cells/ml.

As already explained above, the methods of the invention may further comprise that in step (c) the cell density is maintained by cell bleeding. The cell density referred to in this context is the viable cell density, which may be determined by any method known in the art. For example the calculation governing the cell bleed rate may be based on maintaining the Incyte or Futura biomass capacitance probe value (Hamilton company, Aber instruments) which corresponded to the target VCD, or a daily cell and viability count can be taken off-line via any cell counting device, such as haemocytometer, Vi-Cell (Beckman Coulter), Cedex HiRes (Roche), or Viacount assay (EMD Millipore Guava EasyCyte). Using the methods of the present invention the cell bleeding is reduced compared to a control perfusion cell culture, wherein a control perfusion cell culture is a perfusion cell culture that is cultured under the same conditions using the same serum-free perfusion medium without the Fe ion concentration and the retinoid concentration according to the invention. More specifically the cell bleeding is reduced compared to a control perfusion cell culture, wherein a control perfusion cell culture is a perfusion cell culture that is cultured under the same conditions using the same serum-free perfusion medium with Fe ions at a concentration of less than 230 μM and a retinoid at a concentration of less than 10 μM.

Retinoids have a proliferative and anti-proliferative (inducing growth-arrest) effect, depending on the concentrations. While lower concentrations induce proliferation, higher concentrations inhibit proliferation. Thus the retinoid concentration should not gradually increase in perfusion cell culture to induce growth-arrest in the production phase to avoid unwanted proliferative activity. This can be avoided by adding the retinoid to the perfusion culture in a bolus addition to adjust the retinoid concentration in the mammalian cell culture to the desired retinoid concentration in the serum-free perfusion medium before starting perfusion with the medium of the invention (i.e., prior to step (c)). A bolus addition of retinoid should be added directly to the bioreactor such that the concentration within the reactor will be the desired retinoid concentration instantaneously. The cell bleed rate, controlled based on VCD, should then be decreased as the cells divide more slowly in the presence of retinoid.

A preferred retinoid concentration in the serum-free perfusion medium of the invention is 20 μM to 400 μM, 30 μM to 400 μM 40 μM to 400 μM, 50 μM to 400 μM, about 60 μM to 400 μM, preferably about 100 μM to about 200 μM and more preferably about 150 μM to about 200 μM. The retinoid may be retinol, retinal, retinoic acid or a retinyl ester and is preferably retinol or a retinyl ester. The retinyl ester may be retinyl acetate or retinyl palmitate, preferably retinyl acetate. In one embodiment the retinol or retinyl ester concentration is 10 μM to 400 μM, 20 μM to 400 μM, 30 μM to 400 μM 40 μM to 400 μM, 50 μM to 400 μM, about 60 μM to 400 μM, preferably about 100 μM to about 200 μM and more preferably about 150 μM to about 200 μM. In one embodiment the retinyl ester concentration is 10 μM to 400 μM, 20 μM to 400 μM, 30 μM to 400 μM 40 μM to 400 μM, 50 μM to 400 μM, about 60 μM to 400 μM, preferably about 100 μM to about 200 μM and more preferably about 150 μM to about 200 μM. In one embodiment the retinyl acetate concentration is 10 μM to 400 μM, 20 μM to 400 μM, 30 μM to 400 μM 40 μM to 400 μM, 50 μM to 400 μM, about 60 μM to 400 μM, preferably about 100 μM to about 200 μM and more preferably about 150 μM to about 200 μM. The retinoid concentrations according to the invention lead to a G₀/G₁ cell cycle arrest.

For the same reason and to allow growth during growth phase, the retinoid concentration of the serum-free perfusion medium of the invention should be avoided in the inoculation medium and during growth phase. Thus, the serum-free culture medium of step (a) and the serum-free perfusion medium of step (b) should comprise no retinoid or a retinoid at a concentration lower than the retinoid concentration of the serum-free perfusion medium of the invention. Preferably the serum-free culture medium of step (a) and the serum-free perfusion medium of step (b) comprise no retinoid or less than 1 μM of a retinoid.

Unlike the retinoid, Fe ions can be slowly increased in the mammalian cell culture. It therefore does not need to be added as bolus prior to perfusion with a medium comprising Fe ions at the concentrations according to the invention. Also, since Fe ions do not induce growth arrest, they can be added to the cell culture before production phase is reached, i.e., during growth phase or with the inoculation medium. Thus, the Fe ion concentrations according to the invention may already be present or added with the serum-free perfusion medium in step (b). Furthermore, the Fe ion concentrations according to the invention may already be present or added to the serum-free culture medium of step (a), i.e., the inoculation medium.

A preferred Fe ion concentration according to the present invention is about 350 μM to 3.5 mM, preferably about 1 mM to 3.5 mM, more preferably about 1.4 mM to 3.5 mM and even more preferably about 1.6 mM to 3.5 mM. Alternatively the Fe ion concentration may be in the range of about 350 μM to about 3.1 mM, preferably about 1 mM to about 3.1 mM, more preferably about 1.4 mM to about 3.1 mM and even more preferably about 1.6 mM to about 3.1 mM. The Fe ions may be provided as a ferric (III) or ferrous (II) ions as a salt or in a complexed form. The Fe ion source for use in the present invention may be, without being limited thereto, FePO₄, Fe₄(P₂O₇)₃ pyrophosphate, C₆H₁₀FeO₆, (NH₄)₅[Fe(C₆H₄O₇)₂], FeSO₄, Fe-citrate, Fe-transferrin, iron choline citrate, Fe-EDTA, ethylenediaminetetraacetic acid ferric sodium salt, Fe-dextran, Fe(NO₃)₃, FeCl₂, FeCl₃, or combinations thereof. Preferably the Fe ion source is FeSO₄, Fe-citrate, Fe-transferrin, iron choline citrate, Fe-EDTA, Fe(NO₃)₃, FeCl₂, FeCl₃, or combinations thereof, more preferably FeSO₄, Fe-citrate, Fe(NO₃)₃, Fe-transferrin, or combinations thereof, and even more preferably Fe-citrate.

In one embodiment the Fe ions are provided as FeSO₄, Fe-citrate, Fe-transferrin, iron choline citrate, Fe-EDTA, Fe(NO₃)₃, FeCl₂, FeCl₃, or combinations thereof at a Fe ion concentration of about 350 μM to 3.5 mM, preferably about 1 mM to 3.5 mM, more preferably about 1.4 mM to 3.5 mM and even more preferably about 1.6 mM to 3.5 mM. Alternatively, the Fe ions are provided as FeSO₄, Fe-citrate, Fe-transferrin, iron choline citrate, Fe-EDTA, Fe(NO₃)₃, FeCl₂, FeCl₃, or combinations thereof at a Fe ion concentration of about 350 μM to about 3.1 mM, preferably about 1 mM to about 3.1 mM, more preferably about 1.4 mM to about 3.1 mM and even more preferably about 1.6 mM to about 3.1 mM. In one embodiment the Fe ions are provided as FeSO₄, Fe-citrate, Fe(NO₃)₃, Fe-transferrin, or combinations thereof at a Fe ion concentration of about 350 μM to 3.5 mM, preferably about 1 mM to 3.5 mM, more preferably about 1.4 mM to 3.5 mM and even more preferably about 1.6 mM to 3.5 mM. Alternatively, the Fe ions are provided as FeSO₄, Fe-citrate, Fe(NO₃)₃, Fe-transferrin, or combinations thereof at a Fe ion concentration of about 350 μM to about 3.1 mM, preferably about 1 mM to about 3.1 mM, more preferably about 1.4 mM to about 3.1 mM and even more preferably about 1.6 mM to about 3.1 mM. In one embodiment the Fe ions are provided as Fe-citrate at a concentration of about 350 μM to 3.5 mM, preferably about 1 mM to 3.5 mM, more preferably about 1.4 mM to 3.5 mM and even more preferably about 1.6 mM to 3.5 mM. Alternatively, the Fe ions are provided Fe-citrate at a concentration of about 350 μM to about 3.1 mM, preferably about 1 mM to about 3.1 mM, more preferably about 1.4 mM to about 3.1 mM and even more preferably about 1.6 mM to about 3.1 mM. The person skilled in the art would understand that Fe ions can be ferrous Fe(II) ions as well as ferric Fe(III) ions, e.g., Fe-citrates may be ferrous (iron(II)) (FeC₆H₆O⁷) and ferric (iron (III)) citrate (FeC₆H₅O₇), and is preferably ferric citrate.

Fe ions seem to have a synergistic and/or protective effect with regard to the anti-proliferative or growth-arresting activity of the retinoid, particularly of retinyl acetate. While high concentrations of a retinoid (150 μM to 400 μM) may affect cell viability in the absence of Fe ions or in the presence of low concentrations of Fe ions (<1 mM), cells maintain their viability in the presence of higher Fe ion concentrations (≥1 mM).

In one embodiment the retinoid concentration is, e.g., about 100 μM to about 200 μM, preferably about 150 μM to about 200 μM and the Fe ion concentration is about 1 mM to about 3.1 mM, preferably about 1.4 mM to about 3.1 mM, more preferably about 1.6 mM to about 3.1 mM. In another embodiment the retinoid concentration is, e.g., about 60 μM to about 150 μM, preferably about 100 μM and the Fe ion concentration is about 350 μM to about 1.6 mM, preferably about 350 μM to about 1 mM.

The osmolarity of the serum-free perfusion medium of the invention should be in the range of between 300 and 1400 mOsmol/kg, preferably between 300 and 500 mOsmol/kg, more preferably between 330 and 450 mOsmol/kg and even more preferably between 360 and 390 mOsmol/kg. Wherein the osmolarity is provided as mOsmol/kg water.

The serum-free perfusion medium of the invention and the serum-free perfusion medium used in the methods of the invention may be chemically defined and/or hydrolysate-free. Hydrolysate-free means that the medium does not contain protein hydrolysates from animal, plant (soybean, potato, rice), yeast or other sources. Typically a chemically defined medium is hydrolysate-free. In any case the serum-free perfusion medium should be free of compounds derived from animal sources, particularly proteins or peptides derived and isolated from an animal (this does not include recombinant proteins produced by the cell culture). Preferably the serum-free perfusion medium is protein-free or protein-free except for recombinant insulin and/or insulin-like growth factor. More preferably the serum-free perfusion medium is chemically defined and protein-free or protein-free except for recombinant insulin and/or insulin-like growth factor. This also applies to the serum-free culture medium of step (a) and the serum-free perfusion medium of step (b).

A perfusion culture typically starts with an inoculation culture as batch culture. Perfusion may start immediately or after one or more days. Typically perfusion starts on or after day 2 of the cell culture. In one embodiment the perfusion in step (b) begins on or after day 2 of the cell culture. Once the target cell density is reached growth arrest is induced by increasing the retinoid concentration in the cell culture to obtain the concentration of the serum-free perfusion medium of the invention in the cell culture. Preferably this increase in retinoid concentration to the desired retinoid concentration is instantaneously, such as by bolus addition. Thus, prior to step (c) the retinoid concentration in the mammalian cell culture is adjusted to the retinoid concentration in the serum-free perfusion medium in step (c), preferably by bolus addition, i.e., to a concentration of 10 μM to 400 μM. During production phase the mammalian cells are cultured by perfusion with a serum-free perfusion medium comprising Fe ions at a concentration of 230 μM to 3.5 mM and a retinoid at a concentration of 10 μM to 400 μM. The Fe ion concentration may be added to the culture from the start and hence may be added to the serum-free culture medium used for inoculation or to the serum-free perfusion medium before growth arrest is induced by bolus addition of the retinoid as described above and hence before the target cell density is reached. At the latest it must be added with the serum-free perfusion medium in step (c) once the target cell density is reached with or without a bolus addition of Fe ions to obtain the concentration of the serum-free perfusion medium of the invention in the cell culture. Preferably the desired Fe ion concentration in the mammalian cell culture is reached before the target cell density is reached and before growth arrest is induced by increasing the retinoid concentration in the mammalian cell culture.

Typically the mammalian cell culture according to the invention comprises continuous perfusion of the cell culture. The perfusion rate may increase after perfusion has started. Typically a higher perfusion rate supports a higher viable cell density and therefore allows for a higher target cell density. The perfusion rate may increase from less or equal to 0.5 vessel volumes per day to 5 vessel volumes per day. Preferably the perfusion rate increases from less than or equal to 0.5 vessel volumes per day to 2 vessel volumes per day.

The methods of the present invention further comprise harvesting the heterologous protein from the perfusion cell culture. This is preferably done continuously from the permeate, which is the supernatant produced after cells have been recovered by a cell retention device. Due to the lower product residence time of the product proteins in the cell culture inside the perfusion bioreactor compared to fed-batch, the exposure to proteases, sialidases and other degrading proteins is minimized, which may result in better product quality of heterologous proteins produced in perfusion culture.

According to the methods of the present invention any cell perfusion bioreactor and cell retention device may be used for perfusion culture. The bioreactors used for perfusion are not very different from those used for batch/fed-batch cultures, except that they are more compact in size and are connected to a cell retention device. The methods for retaining cells inside the bioreactor are primarily determined by whether the cells are growing attached to surfaces or growing in either single cell suspension or cell aggregates. While most mammalian cells historically were grown attached to a surface or a matrix (heterogenous cultures), efforts have been made to adapt many industrial mammalian cell lines to grow in suspension (homogenous cultures), mainly because suspension cultures are easier to scale-up. Thus, the cells used in the methods of the invention are preferably grown in suspension. Without being limited thereto, exemplary retention systems for cells grown in suspension are spin filter, external filtration such as tangential flow filtration (TFF), alternating tangential flow (ATF) system, cell sedimentation (vertical sedimentation and inclined sedimentation), centrifugation, ultrasonic separation and hydrocyclones. Perfusion systems can be categorized into two categories, filtration based systems, such as spin filters, external filtration and ATF, and open perfusion systems, such as gravitational settlers, centrifuges, ultrasonic separation devices and hydroclones. Filtration-based systems show a high degree of cell retention and it does not change with the flow rate. However, the filters may clog and hence the cultivation run is limited in length or the filters need to be exchanged. An example for an ATF system is the ATF2 system from Repligen and an example for a TFF system is the TFF system from Levitronix using a centrifugal pump. A cross-flow filter, such as a hollow fiber (HF) or a flat plate filter may be used with ATF and TFF systems. Specifically a hollow fiber, made of modified polyethersulfone (mPES), polyethersulfone (PES), or polysulfone (PE), can be used with ATF and TFF systems. Pore sizes of the HF can range from several hundred kDa to 15 μM. Open perfusion systems do not clog and hence could at least theoretically be operated indefinitely. However, the degree of cell retention is reduced at higher perfusion rates. Currently there are three systems that can be used at industrial scale, alternating tangential filters (ATF), gravitational (particularly inclined settlers) and centrifuges. Cell retention devices suitable for heterogenous or homogenous cultures are described in more detail by Kompala and Ozturk (Cell Culture Technology for Pharmaceutical and Cell-Based Therapies, (2006), Taylor & Francis Group, LLC, pages 387-416). The perfusion culture is not a true steady state process, with the total and viable cell concentration reaching a steady state only when a cell bleed stream is removed from the bioreactor.

Physical parameters such as pH, dissolved oxygen and temperature in a perfusion bioreactor should be monitored on-line and controlled in real time. Determination of cell density, viability, metabolite, and product concentrations may be performed using off-line or on-line sampling. When the perfusion operation starts with continuous harvest and feeding the perfusion rate typically refers to the harvest flow rate, which may be manually set to a desired value. For example, a weight control for the bioreactor may activate the feed pump so that a constant volume in the bioreactor can be maintained. Alternatively, a level control can be achieved by pumping out culture volume above a predetermined level. The perfusion rate in the bioreactor must be adjusted to deliver sufficient nutrients to the cells. As the cell density increases in the bioreactor, the perfusion rate must be increased.

Perfusion rate may be controlled, e.g., using cell density measurements, pH measurements, oxygen consumption or metabolite measurements. Cell density is the most important measurement used for perfusion rate adjustments. Depending on how the cell density measurements are conducted, perfusion rates can be adjusted daily or in real time. Several on-line probes have been developed for the estimation of cell density and are known to the person skilled in the art, such as a capacitance probe, e.g., an Incyte probe (Hamilton Company) or a Futura probe (Aber instruments). These cell density probes can also be used to control the cell density at a desired set point by removing excess cells from the bioreactor, i.e., the cell bleed. Thus, the cell bleed is determined by the specific growth rate of the mammalian cells in culture. The cell bleed is typically not harvested and therefore considered as waste.

In yet another aspect a use of the serum-free perfusion medium of the invention for culturing mammalian cells in a perfusion culture during production phase or for reducing the total cell bleed volume in a perfusion culture is provided. Alternatively, a use of the serum-free perfusion medium of the invention for increasing protein production in a perfusion cell culture is provided. The use of the medium is as disclosed for the serum-free perfusion medium (of step (c)) used in the methods of the inventions.

The heterologous protein produced by the methods and uses of the present invention may be any secreted protein, preferably it is a therapeutic protein. Since most therapeutic proteins are recombinant therapeutic proteins, it is most preferably a recombinant therapeutic protein. Examples for therapeutic proteins are without being limited thereto antibodies, fusion proteins, cytokines and growth factor.

The therapeutic protein produced in the mammalian cells according to the methods of the invention includes, but is not limited to an antibodies or a fusion protein, such as a Fc-fusion proteins. Other secreted recombinant therapeutic proteins can be for example enzymes, cytokines, lymphokines, adhesion molecules, receptors and derivatives or fragments thereof, and any other polypeptides and scaffolds that can serve as agonists or antagonists and/or have therapeutic or diagnostic use.

Other recombinant proteins of interest are for example, without being limited thereto: insulin, insulin-like growth factor, hGH, tPA, cytokines, such as interleukins (IL), e.g. IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, interferon (IFN) alpha, IFN beta, IFN gamma, IFN omega or IFN tau, tumor necrosis factor (TNF), such as TNF alpha and TNF beta, TNF gamma, TRAIL; G-CSF, GM-CSF, M-CSF, MCP-1, and VEGF. Also included is the production of erythropoietin or any other hormone growth factors and any other polypeptides that can serve as agonists or antagonists and/or have therapeutic or diagnostic use.

A preferred therapeutic protein is an antibody or a fragment or derivative thereof, more preferably an IgG1 antibody. Thus, the invention can be advantageously used for production of antibodies such as monoclonal antibodies, multi-specific antibodies, or fragments thereof, preferably of monoclonal antibodies, bi-specific antibodies or fragments thereof. Exemplary antibodies within the scope of the present invention include but are not limited to anti-CD2, anti-CD3, anti-CD20, anti-CD22, anti-CD30, anti-CD33, anti-CD37, anti-CD40, anti-CD44, anti-CD44v6, anti-CD49d, anti-CD52, anti-EGFR1 (HER1), anti-EGFR2 (HER2), anti-GD3, anti-IGF, anti-VEGF, anti-TNFalpha, anti-IL2, anti-IL-5R or anti-IgE antibodies, and are preferably selected from the group consisting of anti-CD20, anti-CD33, anti-CD37, anti-CD40, anti-CD44, anti-CD52, anti-HER2/neu (erbB2), anti-EGFR, anti-IGF, anti-VEGF, anti-TNFalpha, anti-IL2 and anti-IgE antibodies.

Antibody fragments include e.g. “Fab fragments” (Fragment antigen-binding=Fab). Fab fragments consist of the variable regions of both chains, which are held together by the adjacent constant region. These may be formed by protease digestion, e.g. with papain, from conventional antibodies, but similarly Fab fragments may also be produced by genetic engineering. Further antibody fragments include F(ab′)2 fragments, which may be prepared by proteolytic cleavage with pepsin.

Using genetic engineering methods it is possible to produce shortened antibody fragments which consist only of the variable regions of the heavy (VH) and of the light chain (VL). These are referred to as Fv fragments (Fragment variable=fragment of the variable part). Since these Fv-fragments lack the covalent bonding of the two chains by the cysteines of the constant chains, the Fv fragments are often stabilized. It is advantageous to link the variable regions of the heavy and of the light chain by a short peptide fragment, e.g. of 10 to 30 amino acids, preferably 15 amino acids. In this way a single peptide strand is obtained consisting of VH and VL, linked by a peptide linker. An antibody protein of this kind is known as a single-chain-Fv (scFv). Examples of scFv-antibody proteins are known to the person skilled in the art.

Preferred therapeutic antibodies according to the invention are bispecific antibodies. Bispecific antibodies typically combine antigen-binding specificities for target cells (e.g., malignant B cells) and effector cells (e.g., T cells, NK cells or macrophages) in one molecule. Exemplary bispecific antibodies, without being limited thereto are diabodies, BiTE (Bi-specific T-cell Engager) formats and DART (Dual-Affinity Re-Targeting) formats. The diabody format separates cognate variable domains of heavy and light chains of the two antigen binding specificities on two separate polypeptide chains, with the two polypeptide chains being associated non-covalently. The DART format is based on the diabody format, but it provides additional stabilization through a C-terminal disulfide bridge.

Another preferred therapeutic protein is a fusion protein, such as a Fc-fusion protein. Thus, the invention can be advantageously used for production of fusion proteins, such as Fc-fusion proteins. Furthermore, the method of increasing protein producing according to the invention can be advantageously used for production of fusion proteins, such as Fc-fusion proteins.

The effector part of the fusion protein can be the complete sequence or any part of the sequence of a natural or modified heterologous protein or a composition of complete sequences or any part of the sequence of a natural or modified heterologous protein. The immunoglobulin constant domain sequences may be obtained from any immunoglobulin subtypes, such as IgG1, IgG2, IgG3, IgG4, IgA1 or IgA2 subtypes or classes such as IgA, IgE, IgD or IgM. Preferentially they are derived from human immunoglobulin, more preferred from human IgG and even more preferred from human IgG1 and IgG2 Non-limiting examples of Fc-fusion proteins are MCP1-Fc, ICAM-Fc, EPO-Fc and scFv fragments or the like coupled to the CH2 domain of the heavy chain immunoglobulin constant region comprising the N-linked glycosylation site. Fc-fusion proteins can be constructed by genetic engineering approaches by introducing the CH2 domain of the heavy chain immunoglobulin constant region comprising the N-linked glycosylation site into another expression construct comprising for example other immunoglobulin domains, enzymatically active protein portions, or effector domains. Thus, an Fc-fusion protein according to the present invention comprises also a single chain Fv fragment linked to the CH2 domain of the heavy chain immunoglobulin constant region comprising e.g. the N-linked glycosylation site.

In a further aspect a method of producing a therapeutic protein is provided using the methods of the invention and optionally further comprising a step of purifying and formulating the therapeutic protein into a pharmaceutically acceptable formulation.

The therapeutic protein, especially the antibody, antibody fragment or Fc-fusion protein is preferably recovered/isolated from the culture medium as a secreted polypeptide. It is necessary to purify the therapeutic protein from other recombinant proteins and host cell proteins to obtain substantially homogenous preparations of the therapeutic protein. As a first step, cells and/or particulate cell debris are removed from the culture medium. Further, the therapeutic protein is purified from contaminant soluble proteins, polypeptides and nucleic acids, for example, by fractionation on immunoaffinity or ion-exchange columns, ethanol precipitation, reverse phase HPLC, Sephadex chromatography, and chromatography on silica or on a cation exchange resin such as DEAE. Methods for purifying a heterologous protein expressed by mammalian cells are known in the art.

In one embodiment the heterologous protein expressed using the methods of the invention is encoded by one or more expression cassette(s) comprising a heterologous polynucleotide coding for the heterologous protein. The heterologous protein may be placed under the control of an amplifiable genetic selection marker, such as dihydrofolate reductase (DHFR), glutamine synthetase (GS). The amplifiable selection marker gene can be on the same expression vector as the heterologous protein expression cassette. Alternatively, the amplifiable selection marker gene and the heterologous protein expression cassette can be on different expression vectors, but integrate in close proximity into the host cell's genome. Two or more vectors that are co-transfected simultaneously, for example, often integrate in close proximity into the host cell's genome. Amplification of the genetic region containing the secreted therapeutic protein expression cassette is then mediated by adding the amplification agent (e.g., MTX for DHFR or MSX for GS) into the cultivation medium.

Sufficiently high stable levels of a heterologous protein expressed by a mammalian cell may also be achieved, e.g., by cloning multiple copies of the heterologous protein encoding-polynucleotide into an expression vector. Cloning multiple copies of the heterologous protein-encoding polynucleotide into an expression vector and amplifying the heterologous protein expression cassette as described above may further be combined.

### Mammalian Cell Lines

Mammalian cells as used herein are mammalian cells lines suitable for the production of a secreted recombinant therapeutic protein and may hence also be referred to as “host cells”. Preferred mammalian cells according to the invention are rodent cells such as hamster cells. The mammalian cells are isolated cells or cell lines. The mammalian cells are preferably transformed and/or immortalized cell lines. They are adapted to serial passages in cell culture and do not include primary non-transformed cells or cells that are part of an organ structure. Preferred mammalian cells are BHK21, BHK TK-, CHO, CHO-K1, CHO-DXB11 (also referred to as CHO-DUKX or DuxB11), a CHO-S cell and CHO-DG44 cells or the derivatives/progenies of any of such cell line. Particularly preferred are CHO cells, such as CHO-DG44, CHO-K1 and BHK21, and even more preferred are CHO-DG44 and CHO-K1 cells. Most preferred are CHO-DG44 cells. Glutamine synthetase (GS)-deficient derivatives of the mammalian cell, particularly of the CHO-DG44 and CHO-K1 cell are also encompassed. In one embodiment of the invention the mammalian cell is a Chinese hamster ovary (CHO) cell, preferably a CHO-DG44 cell, a CHO-K1 cell, a CHO DXB11 cell, a CHO-S cell, a CHO GS deficient cell or a derivative thereof.

The mammalian cell may further comprise one or more expression cassette(s) encoding a heterologous protein, such as a therapeutic protein, preferably a recombinant secreted therapeutic protein. The host cells may also be murine cells such as murine myeloma cells, such as NSO and Sp2/0 cells or the derivatives/progenies of any of such cell line. Non-limiting examples of mammalian cells which can be used in the meaning of this invention are also summarized in Table 1. However, derivatives/progenies of those cells, other mammalian cells, including but not limited to human, mice, rat, monkey, and rodent cell lines, can also be used in the present invention, particularly for the production of biopharmaceutical proteins.

TABLE 1
Mammalian production cell lines

| Cell line | Order Number |
|---|---|
| NS0 | ECACC No. 85110503 |
| Sp2/0-Ag14 | ATCC CRL-1581 |
| BHK21 | ATCC CCL-10 |
| BHK TK⁻ | ECACC No. 85011423 |
| HaK | ATCC CCL-15 |
| 2254-62.2 (BHK-21 derivative) | ATCC CRL-8544 |
| CHO | ECACC No. 8505302 |
| CHO wild type | ECACC 00102307 |
| CHO-K1 | ATCC CCL-61 |
| CHO-DUKX | ATCC CRL-9096 |
| (═CHO duk⁻, CHO/dhfr^{-,}, CHO-DXB11) | |
| CHO-DUKX 5A-HS-MYC | ATCC CRL-9010 |
| CHO-DG44 | Urlaub G, et al., 1983. |
| | Cell. 33: 405-412. |
| CHO Pro-5 | ATCC CRL-1781 |
| CHO-S | Life Technologies |
| | A1136401; CHO—S is |
| | derived from CHO |
| | variant Tobey et al. |
| | 1962 |
| V79 | ATCC CCC-93 |
| B14AF28-G3 | ATCC CCL-14 |
| HEK 293 | ATCC CRL-1573 |
| COS-7 | ATCC CRL-1651 |
| U266 | ATCC TIB-196 |
| HuNS1 | ATCC CRL-8644 |
| CHL | ECACC No. 87111906 |
| CAP¹ | Wölfel J, et al., 2011. |
| | BMC Proc. |
| | 5(Suppl 8): P133. |
| PER.C6 ® | Pau et al., 2001. Vaccines. |
| | 19: 2716-2721. |
| H4-II-E | ATCC CRL-1548 |
| | ECACC No.87031301 |
| | Reuber, 1961. J. Natl. |
| | Cancer Inst. 26: 891-899. |
| | Pitot HC, et al., 1964. |
| | Natl. Cancer Inst. |
| | Monogr. 13: 229-245. |
| H4-II-E-C3 | ATCC CRL-1600 |
| H4TG | ATCC CRL-1578 |
| H4-II-E | DSM ACC3129 |
| H4-II-Es | DSM ACC3130 |

¹CAP (CEVEC's Amniocyte Production) cells are an immortalized cell line based on primary human amniocytes. They were generated by transfection of these primary cells with a vector containing the functions E1 and pIX of adenovirus 5. CAP cells allow for competitive stable production of recombinant proteins with excellent biologic activity and therapeutic efficacy as a result of authentic human posttranslational modification.

Mammalian cells are most preferred, when being established, adapted, and completely cultivated under serum free conditions, and optionally in media, which are free of any protein/peptide of animal origin. Commercially available media such as Ham's F12 (Sigma, Deisenhofen, Germany), RPMI-1640 (Sigma), Dulbecco's Modified Eagle's Medium (DMEM; Sigma), Minimal Essential Medium (MEM; Sigma), Iscove's Modified Dulbecco's Medium (IMDM; Sigma), CD-CHO (Invitrogen, Carlsbad, Calif.), CHO-S-Invitrogen), serum-free CHO Medium (Sigma), and protein-free CHO Medium (Sigma) are exemplary appropriate nutrient solutions. Any of the media may be supplemented as necessary with a variety of compounds, non-limiting examples of which are recombinant hormones and/or other recombinant growth factors (such as insulin, transferrin, epidermal growth factor, insulin like growth factor), salts (such as sodium chloride, calcium, magnesium, phosphate), buffers (such as HEPES), nucleosides (such as adenosine, thymidine), glutamine, glucose or other equivalent energy sources, antibiotics and trace elements. Any other necessary supplements may also be included at appropriate concentrations that would be known to those skilled in the art. For the growth and selection of genetically modified cells expressing a selectable gene a suitable selection agent is added to the culture medium.

### Serum-Free Perfusion Medium

In another aspect a serum-free perfusion medium is provided comprising Fe ion at a concentration ranging from 230 μM to 3.5 mM and a retinoid in a concentration ranging from 10 μM to 400 μM.

A preferred retinoid concentration in the serum-free perfusion medium of the invention is 20 μM to 400 μM, 30 μM to 400 μM 40 μM to 400 μM, 50 μM to 400 μM, about 60 μM to 400 μM, preferably about 100 μM to about 200 μM and more preferably about 150 μM to about 200 μM. The retinoid may be retinol, retinal, retinoic acid or a retinyl ester and is preferably retinol or a retinyl ester. The retinyl ester may be retinyl acetate or retinyl palmitate, preferably retinyl acetate. In one embodiment the retinol or retinyl ester concentration is 10 μM to 400 μM, 20 μM to 400 μM, 30 μM to 400 μM 40 μM to 400 μM, 50 μM to 400 μM, about 60 μM to 400 μM, preferably about 100 μM to about 200 μM and more preferably about 150 μM to about 200 μM. In one embodiment the retinyl ester concentration is 10 μM to 400 μM, 20 μM to 400 μM, 30 μM to 400 μM 40 μM to 400 μM, 50 μM to 400 μM, about 60 μM to 400 μM, preferably about 100 μM to about 200 μM and more preferably about 150 μM to about 200 μM. In one embodiment the retinyl acetate concentration is 10 μM to 400 μM, 20 μM to 400 μM, 30 μM to 400 μM 40 μM to 400 μM, 50 μM to 400 μM, about 60 μM to 400 μM, preferably about 100 μM to about 200 μM and more preferably about 150 μM to about 200 μM.

A preferred Fe ion concentration according to the present invention is about 350 μM to 3.5 mM, preferably about 1 mM to 3.5 mM, more preferably about 1.4 mM to 3.5 mM and even more preferably about 1.6 mM to 3.5 mM. Alternatively the Fe ion concentration may be in the range of about 350 μM to about 3.1 mM, preferably about 1 mM to about 3.1 mM, more preferably about 1.4 mM to about 3.1 mM and even more preferably about 1.6 mM to about 3.1 mM. The Fe ions may be provided as a ferric (III) or ferrous (II) ions as salt or in a complexed form. The Fe ion source for use in the present invention may be, without being limited thereto, FePO₄, Fe₄(P₂O₇)₃ pyrophosphate, C₆H₁₀FeO₆, (NH₄)₅[Fe(C₆H₄O₇)₂], FeSO₄, Fe-citrate, Fe-transferrin, iron choline citrate, Fe-EDTA, ethylenediaminetetraacetic acid ferric sodium salt, Fe-dextran, Fe(NO₃)₃, FeCl₂, FeCl₃, or combinations thereof. Preferably the Fe ion source is FeSO₄, Fe-citrate, Fe-transferrin, iron choline citrate, Fe-EDTA, Fe(NO₃)₃, FeCl₂, FeCl₃, or combinations thereof, more preferably FeSO₄, Fe-citrate, Fe(NO₃)₃, Fe-transferrin, or combinations thereof, and even more preferably Fe-citrate.

In one embodiment the Fe ions are provided as FeSO₄, Fe-citrate, Fe-transferrin, iron choline citrate, Fe-EDTA, Fe(NO₃)₃, FeCl₂, FeCl₃, or combinations thereof at a Fe ion concentration of about 350 μM to 3.5 mM, preferably about 1 mM to 3.5 mM, more preferably about 1.4 mM to 3.5 mM and even more preferably about 1.6 mM to 3.5 mM. Alternatively the Fe ions are provided as FeSO₄, Fe-citrate, Fe-transferrin, iron choline citrate, Fe-EDTA, Fe(NO₃)₃, FeCl₂, FeCl₃, or combinations thereof at a Fe ion concentration of about 350 μM to about 3.1 mM, preferably about 1 mM to about 3.1 mM, more preferably about 1.4 mM to about 3.1 mM and even more preferably about 1.6 mM to about 3.1 mM. In one embodiment the Fe ions are provided as FeSO₄, Fe-citrate, Fe(NO₃)³, Fe-transferrin, or combinations thereof at a Fe ion concentration of about 350 μM to 3.5 mM, preferably about 1 mM to 3.5 mM, more preferably about 1.4 mM to 3.5 mM and even more preferably about 1.6 mM to 3.5 mM. Alternatively the Fe ions are provided as FeSO₄, Fe-citrate, Fe(NO₃)₃, Fe-transferrin, or combinations thereof at a Fe ion concentration of about 350 μM to about 3.1 mM, preferably about 1 mM to about 3.1 mM, more preferably about 1.4 mM to about 3.1 mM and even more preferably about 1.6 mM to about 3.1 mM. In one embodiment the Fe ions are provided as Fe-citrate at a concentration of about 350 μM to 3.5 mM, preferably about 1 mM to 3.5 mM, more preferably about 1.4 mM to 3.5 mM and even more preferably about 1.6 mM to 3.5 mM. Alternatively the Fe ions are provided as Fe-citrate at a concentration of about 350 μM to about 3.1 mM, preferably about 1 mM to about 3.1 mM, more preferably about 1.4 mM to about 3.1 mM and even more preferably about 1.6 mM to about 3.1 mM. The person skilled in the art would understand that Fe ions means ferrous Fe(II) ions as well as ferric Fe(III) ions, e.g., Fe-citrates may be ferrous (iron(II)) (FeC₆H₆O₇) and ferric (iron (III)) citrate (FeC₆H₅O₇), and is preferably ferric citrate.

Fe ions seem to have a synergistic and protective effect with regard to the anti-proliferative or growth-arresting activity of the retinoid, particularly of retinyl acetate. While high concentrations of a retinoid (150 μM to 400 μM) may affect cell viability in the absence of Fe ions or in the presence of low concentrations of Fe ions (<1 mM), cells maintain their viability in the presence of higher Fe ion concentrations (≥1 mM).

In one embodiment the retinoid concentration is, e.g., about 100 μM to about 200 μM, preferably about 150 μM to about 200 μM and the Fe ion concentration is about 1 mM to about 3.1 mM, preferably about 1.4 mM to about 3.1 mM, more preferably about 1.6 mM to about 3.1 mM. In another embodiment the retinoid concentration is, e.g., about 60 μM to about 150 μM, preferably about 100 μM and the Fe ion concentration is about 350 μM to about 1.6 mM, preferably about 350 μM to about 1 mM.

The osmolarity of the serum-free perfusion medium of the invention should be in the range of between 300 and 1400 mOsmol/kg, preferably between 300 and 500 mOsmol/kg, more preferably between 330 and 450 mOsmol/kg and even more preferably between 360 and 390 mOsmol/kg. Wherein the osmolarity is provided as mOsmol/kg water.

The serum-free perfusion medium of the invention may be chemically defined and/or hydrolysate-free. Hydrolysate-free means that the medium does not contain protein hydrolysates from animal, plant (soybean, potato, rice), yeast or other sources. Typically a chemically defined medium is hydrolysate-free. In any case the serum-free perfusion medium should be free of compounds derived from animal sources, particularly proteins or peptides derived and isolated from an animal (this does not include recombinant proteins produced cell culture). Preferably the serum-free perfusion medium is protein-free or protein-free except for recombinant insulin and/or insulin-like growth factor. More preferably the serum-free perfusion medium is chemically defined and protein-free or protein-free except for recombinant insulin and/or insulin-like growth factor.

In yet another aspect a use of the serum-free perfusion medium of the invention for culturing mammalian cells in a perfusion culture during production phase or for reducing the total cell bleed volume in a perfusion culture is provided. Alternatively a use of the serum-free perfusion medium of the invention for increasing protein production in a perfusion cell culture is provided. The use of the medium is as disclosed for the serum-free perfusion medium (of step (c)) used in the methods of the inventions.

### EXAMPLES

### Example 1

### Deep Well Plate Model of Perfusion

Axygen 24 well pre-sterilized deep-well plates (Neta Scientific Inc, Hainesport, N.J.) were used as a scale-down model of perfusion. A starting cell density of 20×10⁶ cells/mL (3 mL per well) was targeted to simulate the high cell density growth phase of perfusion. Chinese hamster ovary (CHO) DG44 cells expressing a recombinant monoclonal IgG1 antibody were grown in proprietary chemically defined, serum-free perfusion media at 33° C. with 5% CO₂, 80% humidity, and 200 rpm shaking speed in an incubator with a 5.0 cm orbit diameter for 4 or 8 days. A 0.1 mL sample was taken for cell count daily prior to media exchange. Medium exchanges were performed daily by centrifuging the plate at 1200 rpm for 5 minutes, removing 2 mL supernatant, and resuspending cells in 2.1 mL fresh, pre-warmed media at 37° C. (in early days, this equates to a 2/3 volume exchange, whereas in later days the cell pellet grows such that 2 mL is nearly the entire liquid volume, equating to a 1 VVD exchange). Supernatant was saved to perform process analytics.

### Media Preparation

Retinyl acetate was tested in concentrations ranging from 5 μM to 600 μM in proprietary chemically defined, serum-free perfusion medium with 230 μM or 3.1 mM ferric citrate. A 152 mM stock solution of retinyl acetate was prepared by dissolving 50 mg/mL retinyl acetate powder to prepare a 152 mM stock (Sigma-Aldrich; CAS 127-47-9) in absolute ethanol, or 75 mg/mL retinyl acetate for 228 mM stock, for each experimental run; after thorough mixing, the solutions were sterile-filtered with 0.22 μm Steriflips (EMD Millipore). This stock was diluted down further with perfusion medium to provide a working stock. Media aliquot concentrations were 30% higher than the targeted final concentration in each treatment to compensate for residual media left in the well and cell pellet volume. The media were prepared in proprietary chemically defined, serum-free media (either 230 μM or 3.1 mM Fe background) as shown in Table 2. Control condition had no retinyl acetate supplement; ethanol control added the solvent with no retinyl acetate at the level equivalent to the greatest amount for each experiment. These media aliquots were used in the daily media exchanges as described above.

TABLE 2

| Target | | | | |
|---|---|---|---|---|
| retinyl acetate | Concentration | Stock solution | Vol stock | |
| concentration | in medium | concentration | solution | Media volume |
| in well (μM) | added (μM) | (mM) | (μL) | (total; mL) |
| 5 | 7 | 0.040 | 5000 | 40 |
| 20 | 29 | 152 | 7.5 | 40 |
| 40 | 57 | 152 | 15.1 | 40 |
| 80 | 114 | 228 | 25.1 | 50 |
| 100 | 143 | 228 | 31.3 | 50 |
| 200 | 286 | 228 | 62.7 | 50 |
| 400 | 571 | 228 | 125.3 | 50 |
| 600 | 714 | 228 | 188.0 | 50 |

### Process Analytics

Viability and viable cell density were measured by Vi-CELL Cell Counter using the Trypan blue dye exclusion method (Beckman Coulter Life Sciences, Indianapolis, Ind.). Titer, metabolites, and lactate dehydrogenase (LDH) levels were measured using a Cedex BioHT (Roche Diagnostics GmbH, Mannheim, Germany). These titer measurements had been previously verified to be in agreement with in house HPLC Protein A methods. Osmolality was measured with a 2020 Multi-Sample Micro Osmometer (Advanced Instruments, Inc, Norwood, Mass.) based on freezing point depression.

### Cell Cycle Analysis

Cell cycle analysis was performed on days 1 and 7 of deep well culture. Cells were grown in medium containing 3.1 mM ferric citrate and 400 μM retinyl acetate or control medium and fixed on days 1 and 7 by a wash in cold PBS before suspension in 70% ethanol. Cells were stored at −20 ° C. until analysis. 1e5 cells per sample were stained with propidium iodide with RNase (BD Biosciences) for DNA content and analyzed by flow cytometry on a Guava easyCyte flow cytometer (EMD Millipore).

### Results

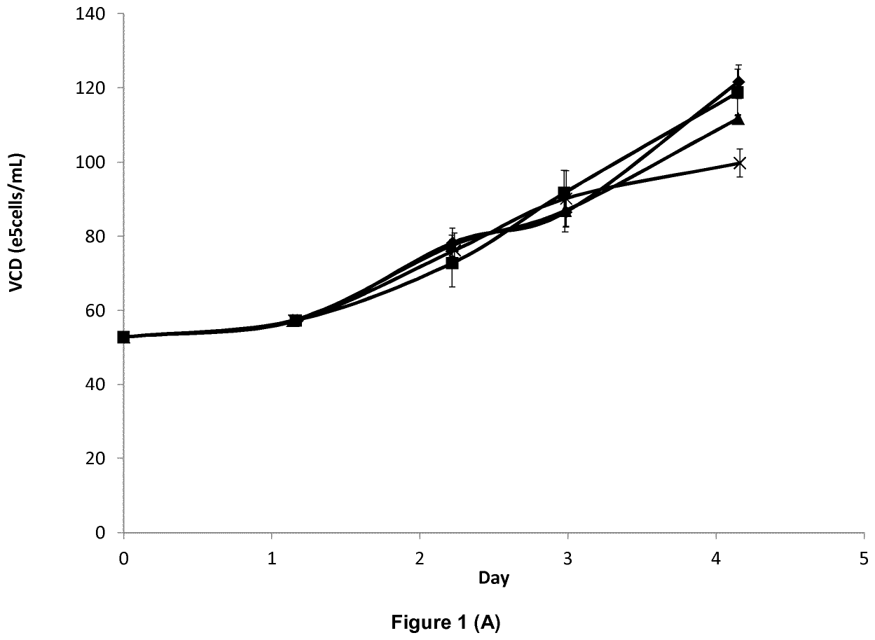

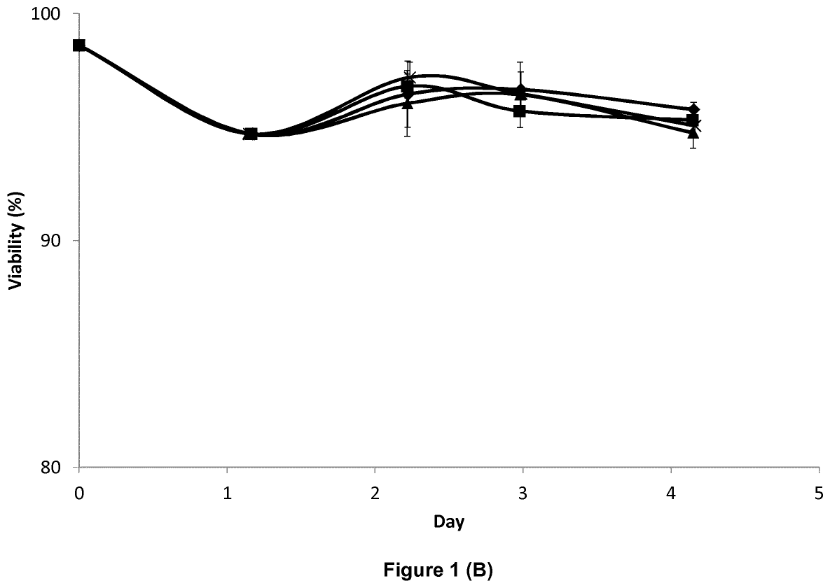

As may be taken from FIG. 1A addition of retinyl acetate at concentrations between 5 μM and 20 μM in the presence of 3.1 mM ferric citrate reduced the viable cell density in a dose-dependent manner, without affecting viability (FIG. 1B).

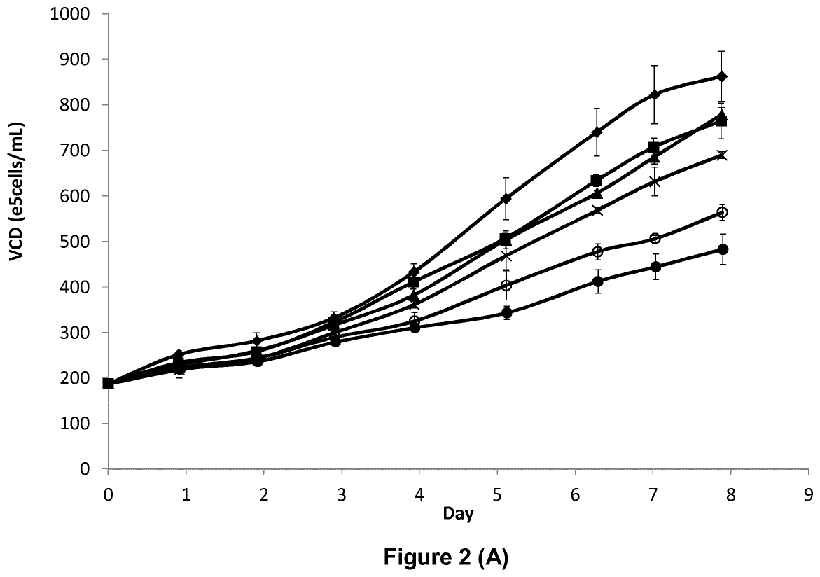

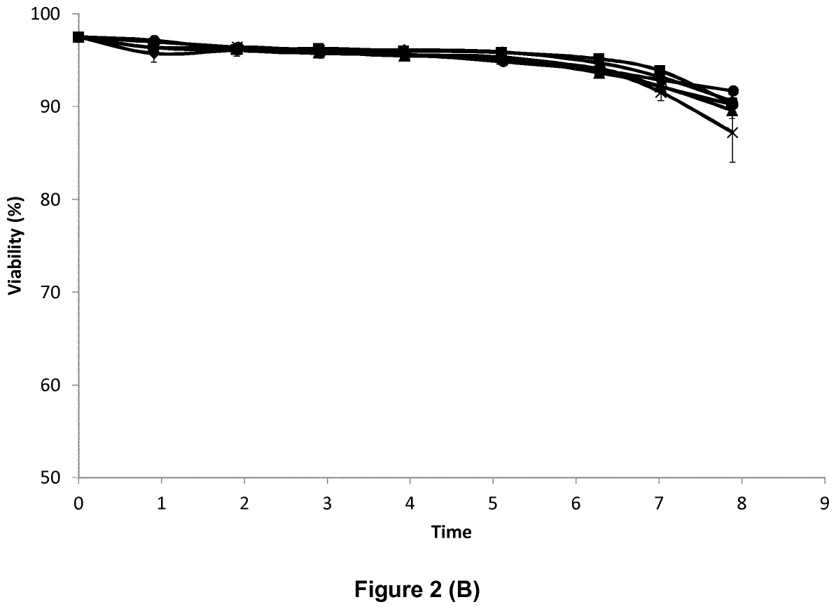

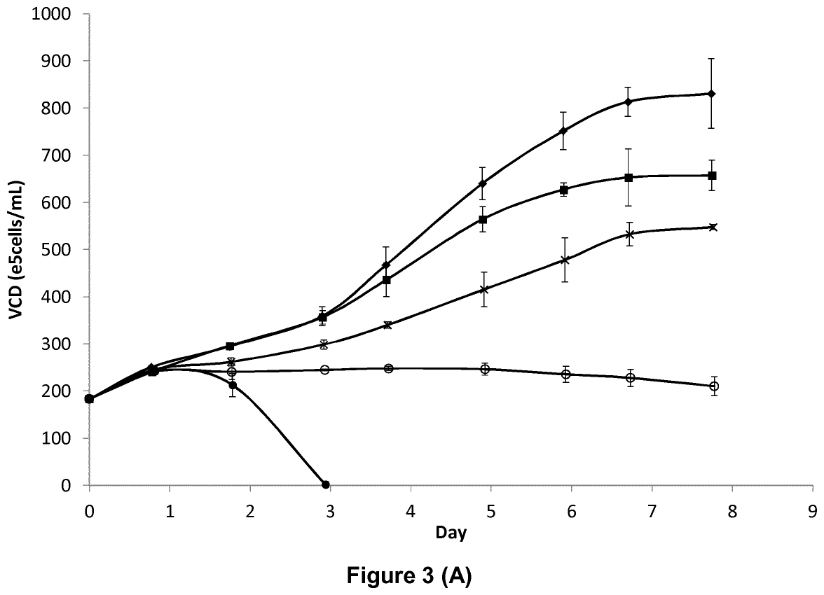

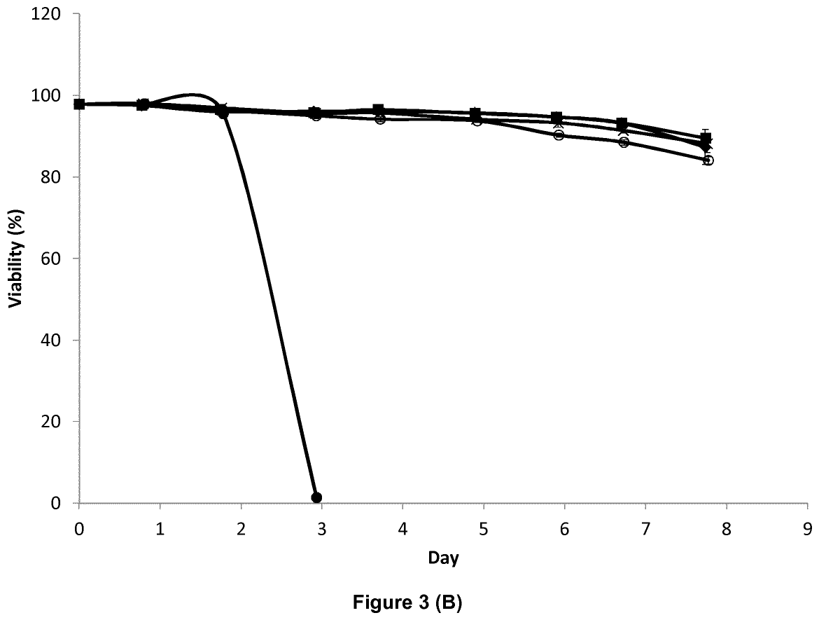

Concentrations of retinyl acetate ranging from 60 μM to 400 μM have been shown to inhibit cell proliferation in the presence of 3.1 mM ferric citrate with no obvious deleterious effect on culture viability in small scale studies. As may be taken from FIG. 2A addition of retinyl acetate at concentrations between 40 μM and 100 μM in the presence of 3.1 mM ferric citrate reduced the viable cell density in a dose-dependent manner, without affecting viability (FIG. 2B). In a separate experiment higher retinyl acetate concentrations (200 μM to 600 μM) were tested in the presence of 3.1 mM ferric citrate. As may be taken from FIG. 3A 200 μM and 400 μM further inhibited growth resulting in a lower viable cell density without effecting viability (FIG. 3B). In cell cultures with 600 μM retinyl acetate the viable cell density sharply declined from about day 2, which is reflected by a strong decrease in cell viability (FIGS. 3A and B). Cell specific productivity (qp) was unaffected (data not shown).

Concentrations from 500-1000 μM caused precipitous declines in viability with this Fe level (FIG. 3). At 2 μM retinyl acetate, no effect on SGR was observed (data not shown). Using culture media comprising 200-400 μM retinyl acetate and ferric citrate at a lower concentration of 230 μM, cell viability was unaffected for a period of time, but started to decline at later time points (data not shown). Further, the effect on viable cell density was present, but less pronounced (data not shown).

Cell cycle analysis revealed that following cultivation for 7 days in a medium comprising 400 μM retinly acetate in the presence of 3.1 mM ferric citrate 86% of cells were in the G0/G1 phase compared to 62.6% of the control cells (which was similar to day 1 in both samples). Thus, retinyl acetate seems to arrest cells in the G0/G1 phase.

### Example 2

### Perfusion Culture

A high density perfusion culture of CHO-DG44 cells expressing a recombinant monoclonal IgG1 antibody was treated with high iron (Fe) and vitamin A (retinyl acetate) to assess the effect on cell proliferation, and by extension, the cell bleed. A bench-scale experiment was performed in three 2L perfusion bioreactors with 0.2 μm PES hollow fibers (Spectrum Labs) on alternating tangential flow (ATF) at 0.8 L/min flow rate. Bioreactors were maintained at: 37° C., 60% dissolved oxygen, 348 RPM agitation, and a target pH of 7.0 (pH target was not always achieved). A CHO DG44 cell line expressing a monoclonal antibody was inoculated into the bioreactors at approximately 1e6 cells/mL in proprietary chemically defined, serum-free inoculation medium at a 1:4 dilution of conditioned to fresh inoculation media. This culture was grown in batch mode (no flow in or out) for 2 days, at which time perfusion was started with BI's perfusion media (manufactured by SAFC). The perfusion rate was gradually increased from 0 to 2 vessel volumes per day (VVD) over 6 days, after which perfusion rate was set at 2 VVD for the duration of the run (Table 3). During the growth phase (d0-d10 post-inoculation) there was no cell bleed, allowing the viable cell density (VCD) to reach desired level. On day 8, ferric citrate (Sigma-Aldrich; CAS 3522-50-7) was added to the perfusion medium at a concentration of 1.6 mM, targeting a pH of 6.9. Once optimal cell density was reached on day 10, cell bleed was initiated to maintain constant VCD (steady state). The calculation governing the cell bleed rate was based on maintaining the Incyte biomass capacitance probe value (Hamilton) which corresponded to the target VCD.

After steady state was established for several days, retinyl acetate (Sigma-Aldrich, CAS 127-47-9) was introduced to two of the bioreactors, with one bioreactor acting as control with no retinyl acetate (Table 4). Retinyl acetate was dissolved in absolute ethanol and sterile-filtered with a 0.2 μm filter to make a sterile stock solution of 152 mM before addition to media bags for final concentrations of 50 μM, 100 μM, 150 μM, or 200 μM retinyl acetate (in four respective bags). Each treatment bioreactor then received retinyl acetate at a first concentration between day 15 and 20 (50 μM or 100 μM) and at a second concentration between days 20-24 (150 or 200 μM) as shown in Table 4. On day 15 and 20 for each treatment, the bag of new treatment media was attached to the media inlet line of the bioreactors. In order to ensure an instantaneous achievement of target retinyl acetate concentration within the vessels (as opposed to the gradual increase which would occur via introduction through the perfusion medium), a bolus addition of retinyl acetate stock solution was added directly to each treatment bioreactor via injection through a sterile addition line (Table 5). The same method was employed when the vitamin level was increased for each treatment vessel.

Samples were taken near-daily for: VCD and viability (Vi-Cell, Beckman-Coulter), osmolality (Advanced Instrument Inc), pH, CO₂, and O₂ (RapidLab 1240 BGA, Siemens), and metabolite readings (Cedex BioHT, Roche). DeltaV was used to control and Finesse TruBio software was used to record and monitor cell bleed, permeate, and media pump rates. Cell samples were taken throughout the experiment and stored at −20 ° C. in 70% EtOH after treatment with Accumax (Innovative Cell Technologies) for later cell cycle analysis using propidium iodide stain with RNase (BD Biosciences) on a Guava easyCyte flow cytometer (EMD Millipore).

TABLE 3
Perfusion rates during growth phase

| | | Vessel volumes | |
|---|---|---|---|
| Day | | per day | Liters per day |
| post-inoculation | Perfusion day | (VVD) | perfused |
| 2 | 0 | 0.50 | 1.0 |
| 3 | 1 | 0.75 | 1.5 |
| 4 | 2 | 1.0 | 2.0 |
| 5 | 3 | 1.3 | 2.5 |
| 6 | 4 | 1.5 | 3.0 |
| 7 | 5 | 2.0 | 4.0 |
| 8 | 6 | 2.0 | 4.0 |

TABLE 4
Treatments of retinyl acetate for each bioreactor

Treatment day

| Bioreactor ID | Day 0-15 | Day 15-20 | | Day 20-24 | |
|---|---|---|---|---|---|
| Control | 0 μM | 0 | μM | 0 | μM |
| X | 0 μM | 50 | μM | 150 | μM |
| Y | 0 μM | 100 | μM | 200 | μM |

TABLE 5
Bolus additions of retinyl acetate stock
solution (152 mM) to each bioreactor.

| | | |
|---|---|---|
| Bioreactor ID | Day 15 | Day 20 |

| | | | | |
|---|---|---|---|---|
| Control | 0 | mL | 0 | mL |
| X | 0.66 | mL | 2.0 | mL |
| Y | 1.3 | mL | 2.6 | mL |

### Results

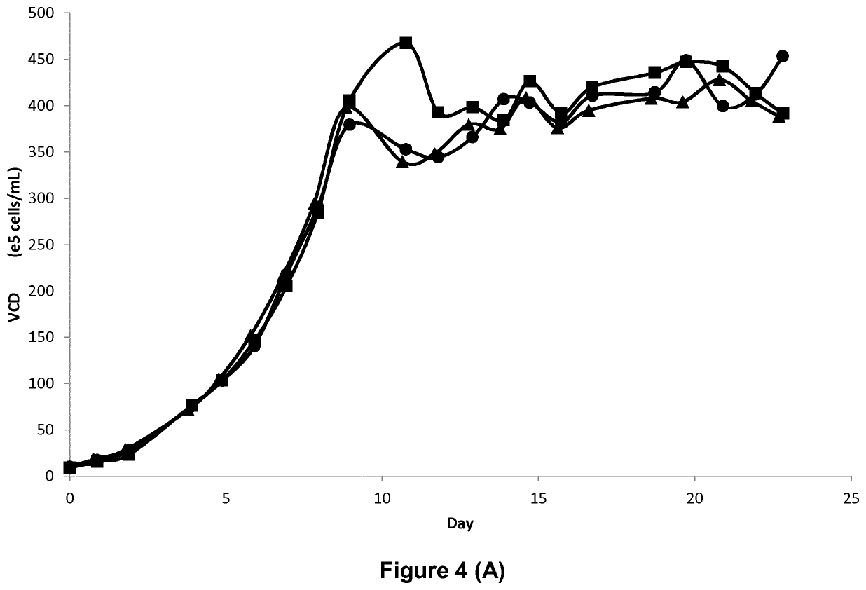

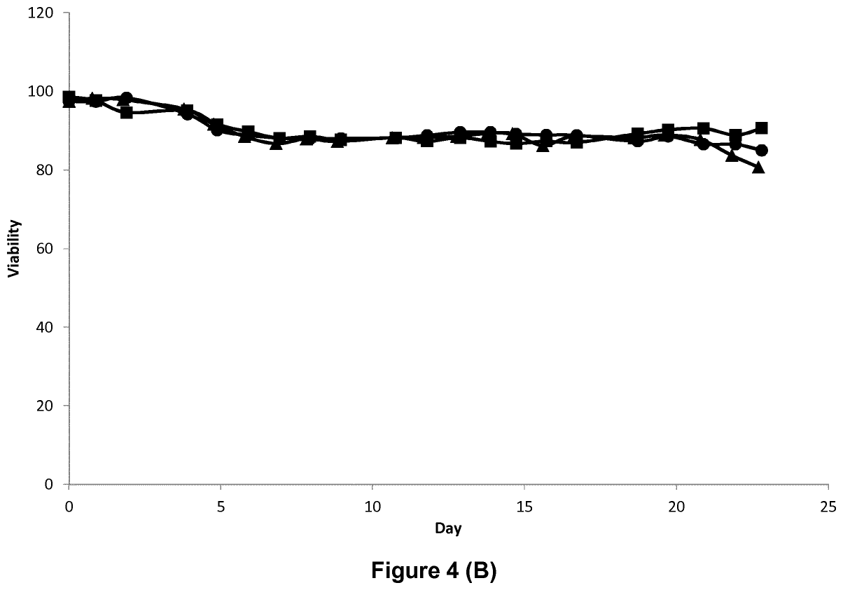

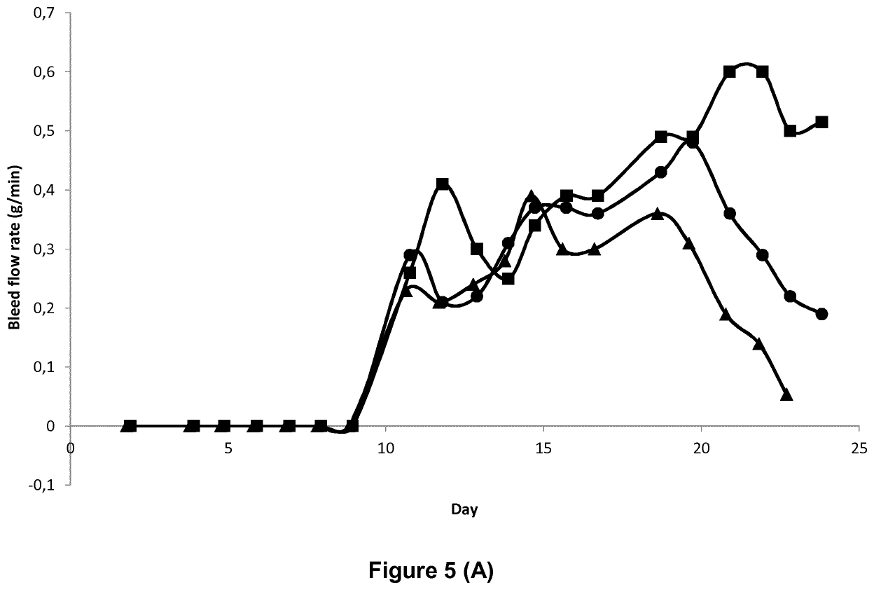

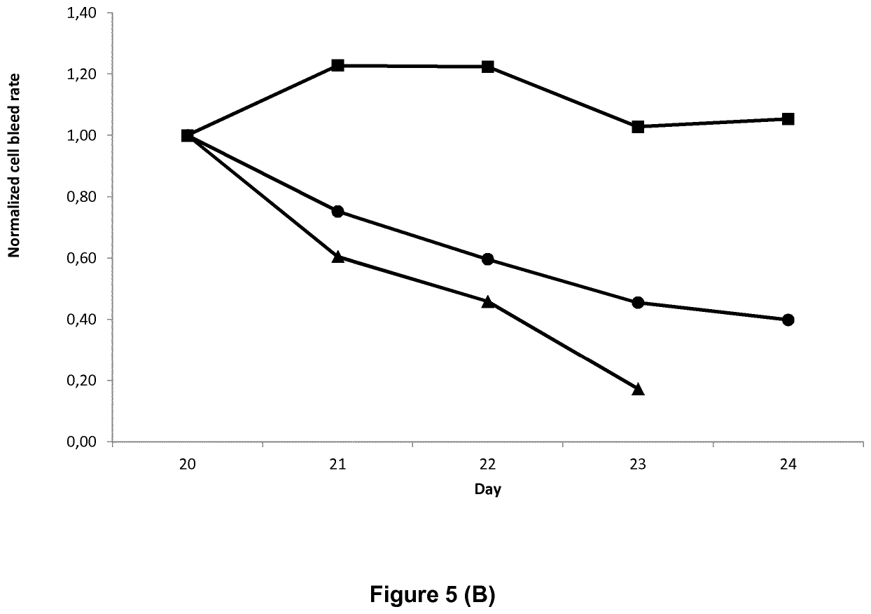

As may be taken from FIG. 4 viable cell density was kept at a constant level and viability was not affected by treatment with 50 μM to 200 μM retinyl acetate in the presence of 1.6 mM ferric citrate. When cultured from days 15 to 20 in a medium containing 1.6 mM ferric citrate and 50 μM retinyl acetate (circles), or 100 μM retinyl acetate (triangles), the bleed flow rate was reduced in a dose-dependent manner compared to control (square) containing ferric citrate at 1.6 mM, but no retinyl acetate (FIG. 5A). The effect was even stronger when cultured from days 20 to 24 in a medium containing 150 μM retinyl acetate (circles) or 200 μM retinyl acetate (triangles) (FIGS. 5A and B). Cell cycle analysis revealed an increase in cell percentage in G0/G1 phase when analyzed following treatment with 150 mM and 200 mM retinyl acetate in the presence of 1.6 mM ferric citrate that increased over time compared to control (data not shown).

### Example 3

A high density perfusion culture of CHO DG44 cells expressing a recombinant monoclonal IgG1 antibody was treated with low iron (Fe) and vitamin A (retinyl acetate) to assess the effect on cell proliferation, and the cell bleed. A bench-scale experiment was performed essentially as described above for Example 2. During the growth phase (d0-d10 post-inoculation) there was no cell bleed, allowing the viable cell density (VCD) to reach desired level. On day 8, ferric citrate (Sigma-Aldrich; CAS 3522-50-7) was added to the perfusion media at a concentration of 350 μM. Once optimal cell density was reached on day 10, cell bleed was initiated to maintain constant VCD (steady state). The calculation governing the cell bleed rate was based on maintaining the Incyte biomass capacitance probe value (Hamilton company) which corresponded to the target VCD.

On day 25 day, retinyl acetate (Sigma-Aldrich, CAS 127-47-9) was introduced to one of the bioreactors, with one bioreactor acting as control with no retinyl acetate. Retinyl acetate was dissolved in absolute ethanol and sterile-filtered with a 0.2 μm filter to make a sterile stock solution of 152 mM before addition to media bags for final concentrations of 100 μM retinyl acetate. Retinyl acetate was then added to the bioreactor on day 25. The bag of new treatment media was attached to the media inlet line of the bioreactors and in order to ensure an instantaneous achievement of target retinyl acetate concentration within the vessels (as opposed to the gradual increase which would occur via introduction through the perfusion medium), a bolus addition of 1.3 mL retinyl acetate stock solution was added directly to the treatment bioreactor via injection through a sterile addition line. Samples were taken and analyzed as explained for Example 2.

### Results

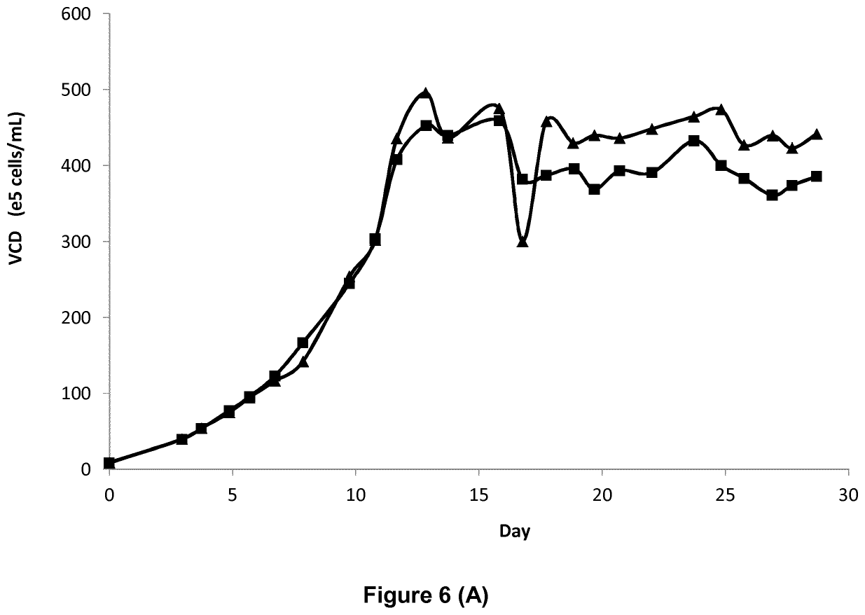

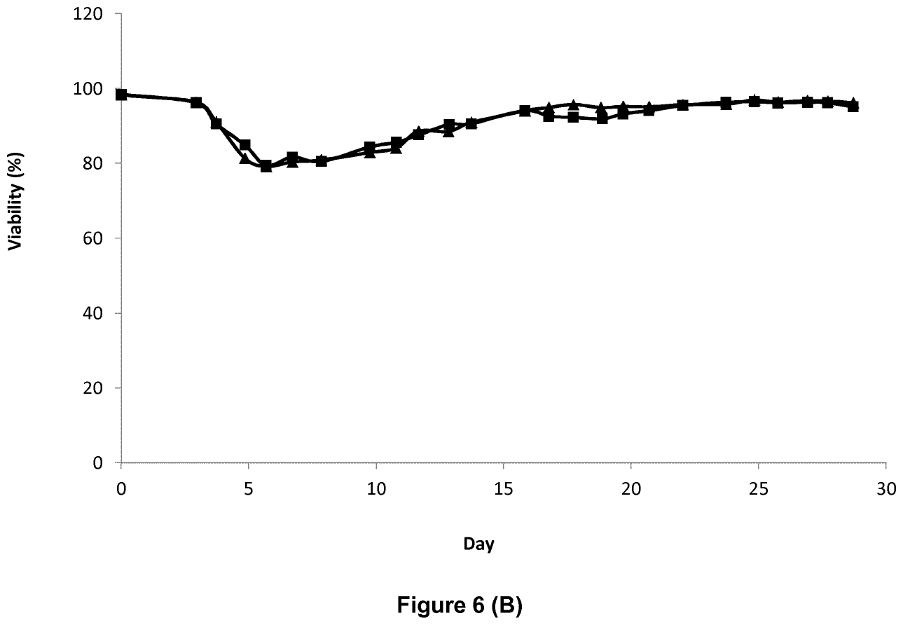

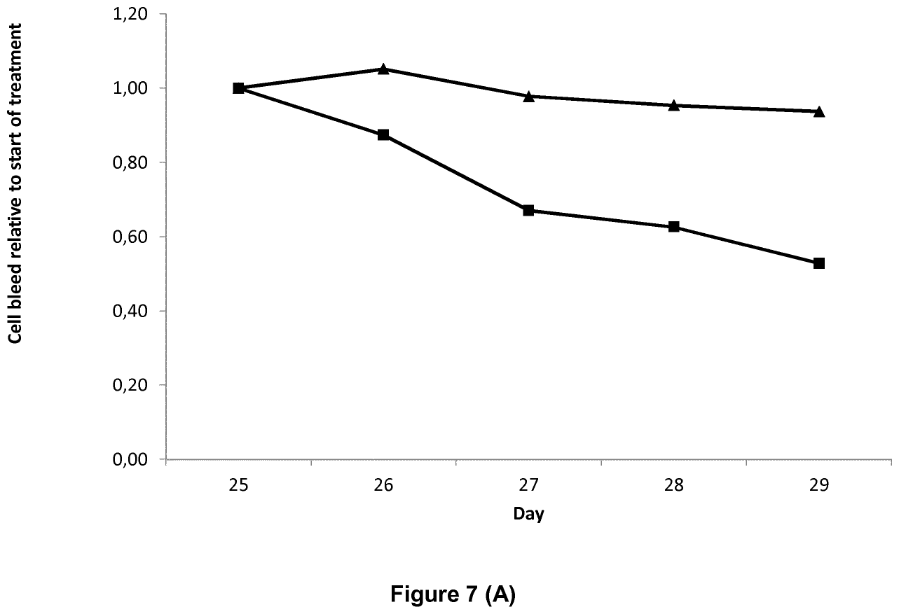

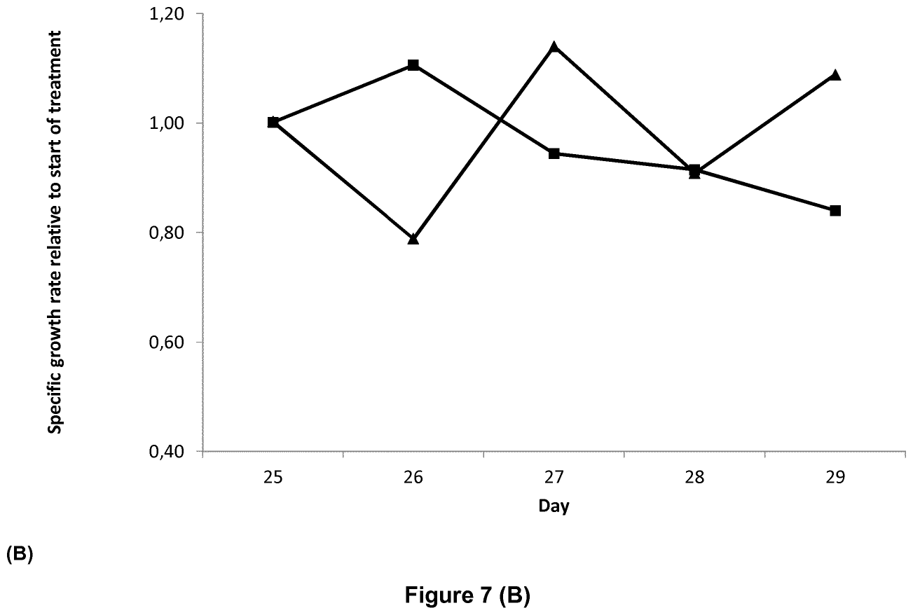

As may be taken from FIG. 6, CHO cells cultured in a medium containing 350 μM ferric citrate and 100 μM retinyl acetate (squares) were maintained at the same viable cell density and had the same high viability as cells treated with 350 μM ferric citrate without retinyl acetate (triangle). However, cell bleed and specific growth rate was markedly decreased during treatment with retinyl acetate (FIG. 7). Treatment with 100 μM vitamin A in 350 μM ferric citrate background media reduced cell bleed rate by approximately 50% over 5 days (FIG. 7A). 150 μM vitamin A in 1.6 mM ferric citrate background media decreased the cell bleed rate by approximately 45% over 4 days (FIG. 5B), and 200 μM decreased it by roughly 70% over the same period (FIG. 5B). The decreases in cell bleed occurred with minimal loss in culture viability.

### Example 4

A small scale cell culture using CHO DG44 cells expressing a recombinant monoclonal IgG1 antibody was treated with ferric citrate and retinyl acetate at different concentrations to assess the effect on cell viability, essentially as described for the small scale culture in Example 1.

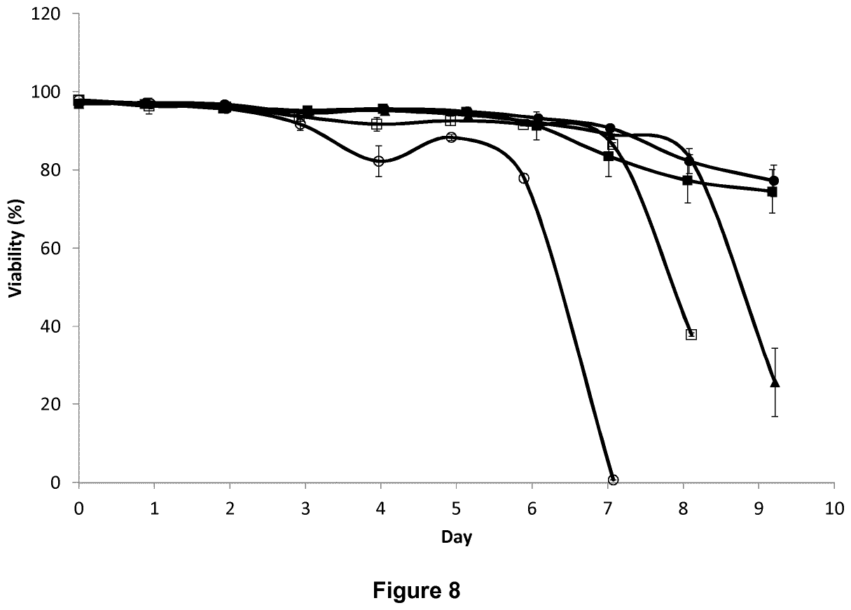

A lower concentration of 230 μM iron has been shown to dampen the CHO growth inhibition response to retinyl acetate; i.e. the decrease in growth rate was less pronounced in low ferric citrate (230 μM) than in high ferric citrate (3.1 mM) for the same retinyl acetate level. Furthermore, the culture appeared to be more sensitized to the cytotoxic effects of retinyl acetate without high concentrations of Fe, as the viability is negatively impacted at retinyl acetate concentrations as low as 200 μM in contrast to higher Fe, where a similar decrease in viability is not observed until 500 μM retinyl acetate (FIG. 8). A decrease in specific growth rate accompanied by a negative effect on viability after 5 days was also observed using a medium comprising 350 μM ferric citrate and 200 μM retinyl acetate (data not shown). In contrast viability was unaffected at the same retinyl acetate concentrations in the presence of 3.1 mM ferric citrate (FIG. 8).

In view of the above, it will be appreciated that the present invention also relates to the following items:

### Items

1. A method of culturing mammalian cells expressing a heterologous protein in a perfusion cell culture comprising:
   (a) culturing mammalian cells expressing a heterologous protein in a serum-free culture medium;
   (b) culturing the mammalian cells during growth phase by perfusion with a serum-free perfusion medium; and
   (c) maintaining the mammalian cells during production phase by perfusion with a serum-free perfusion medium comprising Fe ions at a concentration of 230 μM to 3.5 mM and a retinoid at a concentration of 10 μM to 400 μM,
   wherein step (b) is optional.
2. A method of reducing cell bleeding in a perfusion cell culture expressing a heterologous protein comprising:
   (a) culturing mammalian cells expressing a heterologous protein in a serum-free culture medium;
   (b) culturing the mammalian cells during growth phase by perfusion with a serum-free perfusion medium; and
   (c) maintaining the mammalian cells during production phase by perfusion with a serum-free perfusion medium comprising Fe ions at a concentration of 230 μM to 3.5 mM and a retinoid at a concentration of 10 μM to 400 μM,
   wherein step (b) is optional.
3. A method of increasing protein production in a perfusion cell culture expressing a heterologous protein comprising:
   (a) culturing mammalian cells expressing a heterologous protein in a serum-free culture medium;
   (b) culturing the mammalian cells during growth phase by perfusion with a serum-free perfusion medium; and
   (c) maintaining the mammalian cells during production phase by perfusion with a serum-free perfusion medium comprising Fe ions at a concentration of 230 μM to 3.5 mM and a retinoid at a concentration of 10 μM to 400 μM,
   wherein step (b) is optional.
4. The method of any one of items 1 to 3, wherein
   (i) step (c) further comprises maintaining a cell density by cell bleeding, and/or
   (ii) prior to step (c) the retinoid concentration in the mammalian cell culture is adjusted to the retinoid concentration in the serum-free perfusion medium of step (c), preferably by bolus addition.
5. The method of item 4, wherein cell bleeding is reduced compared to a perfusion cell culture using the same serum-free perfusion medium with Fe ions at a concentration of less than 230 μM and a retinoid at a concentration of less than 10 μM and cultured under the same conditions.
6. The method of any one of the preceding items, wherein further the serum-free perfusion medium in step (b), and optionally also the serum-free culture medium of step (a), comprises Fe ions at a concentration of 230 μM to 3.5 mM.
7. The method of any one of the preceding items, wherein the retinoid concentration is about 60 μM to 400 μM, preferably about 100 μM to about 200 μM and more preferably about 150 μM to about 200 μM.
8. The method of any one of the preceding items, wherein the Fe ion concentration is about 350 μM to 3.5 mM, preferably about 1 mM to 3.5 mM, more preferably about 1.4 mM to 3.5 mM and even more preferably about 1.6 mM to 3.5 mM.
9. The method of item 8, wherein the Fe ion concentration is about 350 μM to about 3.1 mM, preferably about 1 mM to about 3.1 mM, more preferably about 1.4 mM to about 3.1 mM and even more preferably about 1.6 mM to about 3.1 mM.
10. The method of any one of items 1 to 7, wherein the retinoid concentration is about 100 μM to about 200 μM, preferably about 150 μM to about 200 μM and the Fe ion concentration is about 1 mM to about 3.1 mM, preferably about 1.4 mM to about 3.1 mM, more preferably about 1.6 mM to about 3.1 mM.
11. The method of any one of items 1 to 7, wherein the retinoid concentration is about 60 μM to about 150 μM, preferably about 100 μM and the Fe ion concentration is about 350 μM to about 1.6 mM, preferably about 350 μM to about1 mM.
12. The method of any one of the preceding items, wherein the retinoid is retinol, retinal, retinoic acid or a retinyl ester, preferably retinol or a retinyl ester.
13. The method of item 12, wherein the retinoid is a retinyl ester, preferably retinyl acetate or retinyl palm itate.
14. The method of item 13, wherein the retinoid is retinyl acetate.
15. The method of any one of the preceding items, wherein the Fe ion is provided as FeSO₄, Fe-citrate, Fe-transferrin, iron choline citrate, Fe-EDTA, Fe(NO₃)₃, FeCl₂, FeCl₃, or combinations thereof.
16. The method of any one of the preceding items, wherein the Fe ion is provided as FeSO₄, Fe-citrate, Fe(NO₃)₃, Fe-transferrin, or combinations thereof.
17. The method of item 16, wherein the Fe ion is provided as Fe-citrate.
18. The method of any one of the preceding items, wherein the mammalian cell is a Chinese hamster ovary (CHO) cell.
19. The method of item 18, wherein the CHO cell is a CHO-DG44 cell, a CHO-K1 cell, a CHO DXB11 cell, a CHO-S cell, a CHO GS deficient cell or a derivative of any of these cells.
20. The method of any one of the preceding items, wherein the serum-free culture medium of step (a) and the serum-free perfusion medium of step (b) comprise 0 to less than 1 μM of a retinoid.
21. The method of any one of the preceding items, wherein perfusion in step (b) begins on or after day 2 of the cell culture.
22. The method of any one of the preceding items wherein the perfusion comprises continuous perfusion.
23. The method of any one of the preceding items, wherein the perfusion rate increases after perfusion has started.
24. The method of item 23, wherein the perfusion rate increases from less or equal to 0.5 vessel volumes per day to 5 vessel volumes per day.
25. The method of item 24, wherein the perfusion rate increases from less or equal to 0.5 vessel volumes per day to 2 vessel volumes per day.
26. The method of any one of the preceding items, further comprising harvesting the heterologous protein from the perfusion cell culture.
27. The method of any one of the preceding items, wherein the heterologous protein is a therapeutic protein.
28. The method of item 27, wherein the therapeutic protein is selected from the group consisting of an antibody, a fusion protein, a cytokine and a growth factor.
29. The method of any one of the preceding items, wherein the serum-free perfusion medium is chemically defined.
30. The method of any one of the preceding items, wherein the serum-free perfusion medium is hydrolysate-free.
31. The method of any one of the preceding items, wherein the serum-free perfusion medium is protein-free except for recombinant insulin and/or insulin-like growth factor.
32. The method of any one of items 1 to 30, wherein the serum-free perfusion medium is protein-free.
33. The method of any one of items 1 to 32, wherein step (c) is started once a cell density of 10×10⁶ cells/ml to about 120×10⁶ cells/ml is reached.
34. A method of producing a therapeutic protein using the method of any one of items 1 to 33.
35. The method of item 34, wherein the heterologous protein expressed by the mammalian cell is a therapeutic protein and wherein the therapeutic protein is purified and formulated into a pharmaceutically acceptable formulation.
36. A serum-free perfusion medium comprising Fe ions at a concentration ranging from 230 μM to 3.5 mM and a retinoid in a concentration ranging from 10 μM to 400 μM.
37. The serum-free perfusion medium of item 36, wherein the retinoid concentration is about 60 μM to 400 μM, preferably about 100 μM to about 200 μM and more preferably about 150 μM to about 200 μM.
38. The serum-free perfusion medium of item 36 or 37, wherein the Fe ion concentration is about 350 μM to 3.5 mM, preferably about 1 mM to 3.5 mM, more preferably about 1.4 mM to 3.5 mM and even more preferably about 1.6 mM to 3.5 mM.
39. The serum-free perfusion medium item 38, wherein the Fe ion concentration is about 350 μM to about 3.1 mM, preferably about 1 mM to about 3.1 mM, more preferably about 1.4 mM to about 3.1 mM and even more preferably about 1.6 mM to about 3.1 mM.
40. The serum-free perfusion medium of any one of items 36 to 39, wherein the retinoid concentration is about 100 μM to about 200 μM, preferably about 150 μM to about 200 μM and the Fe ion concentration is about 1 mM to about 3.1 mM, preferably about 1.4 mM to about 3.1 mM, more preferably about 1.6 mM to about 3.1 mM.
41. The serum-free perfusion medium of any one of items 36 to 39, wherein the retinoid concentration is about 60 μM to about 150 μM, preferably about 100 μM and the Fe ion concentration is 350 μM to 1.6 mM, preferably 350 μM to 1 mM.
42. The serum-free perfusion medium of any one of items 36 to 41, wherein the retinoid is retinol, retinal, retinoic acid or a retinyl ester, preferably retinol or a retinyl ester.
43. The serum-free perfusion medium of item 42, wherein the retinoid is a retinyl ester, preferably retinyl acetate or retinyl palm itate.
44. The serum-free perfusion medium of item 43, wherein the retinoid is retinyl acetate.
45. The serum-free perfusion medium of any one of items 36 to 44, wherein the Fe ion is provided as FeSO₄, Fe-citrate, Fe-transferrin, iron choline citrate, Fe-EDTA, Fe(NO₃)₃, FeCl₂, FeCl₃, or combinations thereof.
46. The serum-free perfusion medium of item 45, wherein the Fe ion is provided as FeSO₄, Fe-citrate, Fe(NO₃)₃, Fe-transferrin or combinations thereof.
47. The serum-free perfusion medium of item 46, wherein the Fe ion is provided as Fe-citrate.
48. The serum-free perfusion medium of any one of items 36 to 47, wherein the serum-free perfusion medium is chemically defined.
49. The serum-free perfusion medium of any one of items 36 to 48, wherein the serum-free perfusion medium is hydrolysate-free.
50. The serum-free perfusion medium of any one of items 36 to 49, wherein the serum-free perfusion medium is protein-free except for recombinant insulin and/or insulin-like growth factor.
51. The serum-free perfusion medium of any one of items 36 to 49, wherein the serum-free perfusion medium is protein-free.
52. Use of the serum-free perfusion medium of any one of items 36 to 51 for culturing mammalian cells in a perfusion culture during production phase.
53. Use of the serum-free perfusion medium of any one of items 36 to 51 for reducing the total cell bleed volume in a perfusion culture.
54. Use of the serum-free perfusion medium of any one of items 36 to 51 for increasing protein production in a perfusion cell culture.

1. A method of culturing mammalian cells expressing a heterologous protein in a perfusion cell culture comprising:
(a) culturing mammalian cells expressing a heterologous protein in a serum-free culture medium;
(b) culturing the mammalian cells during growth phase by perfusion with a serum-free perfusion medium; and
(c) maintaining the mammalian cells during production phase by perfusion with a serum-free perfusion medium comprising Fe ions at a concentration of 230 μM to 3.5 mM and a retinoid at a concentration of 10 μM to 400 μM,
wherein step (b) is optional.

2. A method of reducing cell bleeding in a perfusion cell culture expressing a heterologous protein comprising:
(a) culturing mammalian cells expressing a heterologous protein in a serum-free culture medium;
(b) culturing the mammalian cells during growth phase by perfusion with a serum-free perfusion medium; and
(c) maintaining the mammalian cells during production phase by perfusion with a serum-free perfusion medium comprising Fe ions at a concentration of 230 μM to 3.5 mM and a retinoid at a concentration of 10 μM to 400 μM,
wherein step (b) is optional.

3. A method of increasing protein production in a perfusion cell culture expressing a heterologous protein comprising:
(a) culturing mammalian cells expressing a heterologous protein in a serum-free culture medium;
(b) culturing the mammalian cells during growth phase by perfusion with a serum-free perfusion medium; and
(c) maintaining the mammalian cells during production phase by perfusion with a serum-free perfusion medium comprising Fe ions at a concentration of 230 μM to 3.5 mM and a retinoid at a concentration of 10 μM to 400 μM,
wherein step (b) is optional.

4. The method of claim 1, wherein
(iii) step (c) further comprises maintaining a cell density by cell bleeding, and/or
(iv) prior to step (c) the retinoid concentration in the mammalian cell culture is adjusted to the retinoid concentration in the serum-free perfusion medium of step (c), optionally by bolus addition.

5. The method of claim 1, wherein the retinoid is retinol, retinal, retinoic acid or a retinyl ester.

6. The method of claim 1, wherein the Fe ion is provided as FeSO4, Fe-citrate, Fe-transferrin, iron choline citrate, Fe-EDTA, Fe(NO3)3, FeCl2, FeCl3, or combinations thereof.

7. The method of claim 1, wherein the mammalian cell is a Chinese hamster ovary (CHO) cell, preferably a CHO-DG44 cell, a CHO-K1 cell, a CHO DXB11 cell, a CHO-S cell, a CHO GS deficient cell or a derivative of any of these cells.

8. The method of claim 1, further comprising harvesting the heterologous protein from the perfusion cell culture.

9. The method of claim 8, wherein the heterologous protein is a therapeutic protein, selected from the group consisting of an antibody, a fusion protein, a cytokine and a growth factor.

10. The method of claim 1, wherein the serum-free perfusion medium is chemically defined.

11. A method of producing a therapeutic protein using the method claim 1.

12. A serum-free perfusion medium comprising Fe ions at a concentration ranging from 230 μM to 3.5 mM and a retinoid in a concentration ranging from 10 μM to 400 μM.

13. A method of claim 12 for culturing mammalian cells in a perfusion culture during production phase, wherein the method comprises the use of the serum-free perfusion medium of claim 12.

14. A method for reducing the total cell bleed volume in a perfusion culture, wherein the method comprises the use of the serum-free perfusion medium of claim 12.

15. A method for increasing protein production in a perfusion cell culture, wherein the method comprises the use of the serum-free perfusion medium of claim 12.
